(19) United States
(12) Patent Application Publication
(10) Pub. No.: US 2025/0161404 A1
(43) Pub. Date: May 22, 2025

(54) METHODS AND COMPOSITIONS FOR THE CONTROL OF INFLAMMATION IN SKIN DISEASE

(71) Applicants: VANDERBILT UNIVERSITY, Nashville, TN (US); THE UNITED STATES AS REPRESENTED BY THE DEPARTMENT OF VETERANS AFFAIRS, Washington, DC (US)

(72) Inventors: Jack Jacek HAWIGER, Nashville, TN (US); Jozef ZIENKIEWICZ, Nashville, TN (US); Yan Liu, Nashville, TN (US); Huan Qiao, Nashville, TN (US)

(21) Appl. No.: 18/939,615

(22) Filed: Nov. 7, 2024

Related U.S. Application Data
(60) Provisional application No. 63/596,827, filed on Nov. 7, 2023.

(51) Int. Cl. A61K 38/17; A61K 9/00; A61K 45/06; A61P 17/00; G01N 33/68

(52) U.S. Cl. CPC A61K 38/17; A61K 9/0014; A61K 45/06; A61P 17/00; G01N 33/6803; G01N 33/6893

(57) ABSTRACT

Disclosed are methods for assessing the responsiveness of a patient to a Nuclear Transport Checkpoint Inhibitor (NTCI) as well as the efficacy of a NTCI treatment regimen. Additionally disclosed herein are methods of genomic diagnosing a subject with an inflammatory skin disease.

### II. REFERENCE TO SEQUENCE LISTING

The sequence listing submitted on Feb. 12, 2025, as an .XML file entitled “10644-171US1_ST26.xml” created on Jan. 8, 2025, and having a file size of 69, 127 bytes is hereby incorporated by reference pursuant to 37 C.F.R. § 1.52 (e) (5).

### III. CROSS-REFERENCE TO RELATED APPLICATIONS

This application claims the benefit of U.S. Application Provisional No. 63/596,827, filed on Nov. 7, 2023, which is incorporated herein by reference in its entirety.

### I. STATEMENT OF GOVERNMENT SUPPORT

This invention was made with government support under Grant No. BX002750 awarded by the United States Department of Veterans Affairs and Grant No. CA068485 awarded by the National Institutes of Health. The government has certain rights in the invention.

### IV. BACKGROUND

Atopic Dermatitis (AD), also known as eczema, is the most common, recurrent inflammatory skin disorder afflicting an estimated 10-20% of children and 5% of adults in all racial and ethnic groups in the US and abroad, with the highest incidence among African Americans. Therefore, the impact of AD on the well-being of a diverse population is significant and indicative of a need for effective and safe therapies. AD, caused by allergic insults, is mediated by inflammation and manifested by intense itch, recurrent eczematous lesions, and a fluctuating course.

Treatment of AD mediated by a plethora of genes encoding the mediators of skin inflammation poses a significant challenge. Currently used broad spectrum therapy with glucocorticoids (e.g., triamcinolone), and targeted therapies with monoclonal antibodies (e.g., dupilumab, tralokinumab) blocking IL-4 receptor, or small molecule inhibitors targeting intracellular signaling intermediates, such as Janus Kinases (JAKs; e.g., upadacitinib and abrocitinib), Phosphodiesterase 4 (PDE4; e.g., crisaborole), and calcineurin (e.g., tacrolimus, pimecrolimus), only partially inhibit inflammatory signaling responsible for AD development and persistence. Targeting one or two mediators of skin inflammation by inhibitors of JAKs, PDE4, calcineurin, as well as the receptor for IL-4 and IL-13 by monoclonal antibodies, has advanced the treatment of AD and other skin inflammatory diseases. However, other untargeted mediators contribute to the refractoriness or relapse of AD (see FIG. 7). In turn, a broad-spectrum therapy with glucocorticoids through their binding to the cognate nuclear receptor reprograms the inflammatory regulome in the cell's nucleus while simultaneously enhancing the expression of metabolic genes causing hyperglycemia, hyperlipidemia, osteoporosis, and skin atrophy. Thus, there is a critical need for more effective therapeutics for preventing and treating inflammation-mediated diseases and in particular, skin diseases such as atopic dermatitis.

### V. SUMMARY

Disclosed are methods and compositions related to diagnosing and treating inflammatory skin disorders.

In one aspect, disclosed herein are methods of diagnosing an inflammatory skin disease or inflammatory skin disorder caused by a microbial, allergic, autoimmune, constitutive, metabolic, neoplastic and/or physical insults (such as, for example, atopic dermatitis or surgical wounds or burns) in a subject the methods comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates the subject has a skin disease or inflammatory skin disorder.

Also disclosed herein are methods of treating/inhibiting/reducing an inflammatory skin disease or inflammatory skin disorder caused by a microbial, allergic, autoimmune, constitutive, metabolic, neoplastic and/or physical insults (such as, for example, atopic dermatitis) in a subject the method comprising a) obtaining a tissue sample from the subject; b) measuring the tissue sample for elevated expression or activity of one or more of TSLP, IL-1ß, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to the subject with elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111, a therapeutically effective amount of a composition comprising a Nuclear Transport Checkpoint Inhibitor (NTCI), also known as Nuclear Transport Modifier (NTM).

In one aspect, disclosed herein are methods of assessing a treatment regimen or a subjects responsiveness to a treatment regimen for an inflammatory skin disease or inflammatory skin disorder caused by a microbial, allergic, autoimmune, constitutive, metabolic, neoplastic and/or physical insults (such as, for example, atopic dermatitis) in a subject the method comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates that the treatment regimen is not effective against the inflammatory skin disease or inflammatory skin disorder caused by a microbial, allergic, autoimmune, constitutive, metabolic, neoplastic and/or physical insults or the subject is not responsive to the treatment regimen.

Also disclosed herein are methods of any preceding aspect, wherein the treatment regimen comprises a composition comprising a nuclear transport modifier (NTM) (such, as for example, a nuclear transport checkpoint inhibitor (NTCI) including, but not limited to compositions wherein the NTM comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

In one aspect, disclosed herein are methods of any preceding aspect, wherein the inflammatory skin disease or inflammatory skin disorder is caused by microbial agents, autoimmune disorder, alloimmune disorder, allergens (i.e., allergic process), autoinflammatory disorder, metabolic process, neoplastic/oncogenic disorder, or physical insults that are mediated by inflammation (such as, for example, atopic dermatitis).

### VI. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a graphic depiction of Experimental Atopic Dermatitis and Treatment Protocol. Atopic dermatitis (AD)-like phenotype was induced in 8-week-old C57BL/6 female mice by topical application of MC903 (calcipotriol), an analog of vitamin D3. On day 1, an initial bolus dose of 6 nmoles of MC903 was applied topically to the right ear (3 nmoles on each side of the ear). On day 3, dosing once a day (Q.D.) with 2 nmoles of MC903 (1 nmole on each side of the right ear) was begun and continued for 3 weeks. On day 20, a 9-day twice-daily (B.I.D.) topical treatment with 66 nmoles NTCI (cSN50.1 peptide, 33 nmoles on each side of the right ear), or an equal volume of NTCI vehicle (10 μl saline on each side of the right ear) was initiated. Mice were weighed and ear thickness was measured every 2-4 days. Ear samples were collected on day 29 for gene expression and Immunohistochemistry (IHC) analyses. Mice in the Mock Control group were only subjected to the measurements of body weight and the ear thickness.

FIGS. 2A, 2B, and 2C show that NTCI treatment reduces signs of MC903-induced Atopic Dermatitis (AD)-like phenotype manifested by swelling, redness, and scaling. Topical NTCI treatment accelerates healing in an experimental model of AD-like phenotype. Cardinal signs of inflamed skin, swelling (A), and redness and scaling (C) were significantly reduced by topical application of cSN50.1 peptide. FIG. 2A shows ear thickness data is displayed as mean±S.E.M. (n=3 for Mock Control and n=10 for all other conditions). Statistical significance of the difference between ear thickness in NTCI-treated mice (MC903+cSN50.1) and untreated (MC903 Untreated) or saline-treated (MC903+saline) groups was determined by two-way ANOVA using corrected Holm-Sidak test for multiple comparison, *p<0.05, **p<0.005, ***p<0.0005. FIG. 2B shows body weight was not significantly affected by MC903 challenge, NTCI, or by saline treatment albeit body weight gain was arrested in these groups. FIG. 2C shows representative pictures of mice from the Mock Control group taken at the end of experiment and from the cSN50.1-treated group immediately before (MC903 AD Induction) and after NTCI treatment (MC903+cSN50.1).

FIG. 3 shows that the expression of genes encoding the mediators of skin inflammation is suppressed by NTCI. The expression of genes encoding the mediators of allergic inflammation in the ear samples (see Methods and FIG. 1 for a depiction of AD-like phenotype induction and treatment protocol) was determined using a real-time quantitative reverse transcription PCR (qRT PCR). The relative levels of expression were established using Livak's methods (2^{-ΔΔCt}) with the 18S gene as a reference and the Mock Control (unchallenged ear) group as the calibrator. Data is presented as a mean+S.E.M. (n=3 for Mock Control, n=5 for all other conditions). A statistical analysis was performed using an ordinary one-way ANOVA with an uncorrected Fisher's LSD test for a multiple comparison, *p<0.05, **p<0.005, ***p<0.0005. The significance levels displayed over the bars of MC903+Saline and MC903+cSN50.1 columns represent the statistical difference of each compared to MC903 Untreated samples.

FIGS. 4A and 4B show that the attenuation of MC903-induced skin inflammation in the murine pinna by NTCI (cSN50.1 peptide) treatment. FIG. 4A shows exposure to MC903 without treatment or treated with a saline control leads to cellular infiltration comprising predominately macrophages (Iba-1) and eosinophiles (major basic protein or MBP) combined with an influx of CD4+T cells (CD4), leading to the thickening of the pinna as compared to the mock control. Moreover, the significant immunodetection of Ki67-positive cells indicated increased cellular proliferation in the basal cell layers of the epidermis in response to MC903. Treatment with NTCI (cSN50.1 peptide) reduced the cellular mediators of the inflammatory response. NTCI reduced skin edema (see also FIG. 2A). Shown are representative images of pinna samples with immunohistochemistry for key inflammatory cell markers (×20 magnification). FIG. 4B shows a semiquantitative analysis of immunohistochemical (IHC) staining presented in panel A. Scoring was conducted according to following formulas: IHC scoring (all inflammatory cell markers): 0-No or almost undetectable immunoreactivity (IR); 1-Scattered and rare IR in the dermis; 2-Multifocal and barely coalescing IR in the dermis; 3-Diffuse IR in the dermis. IHC scoring for Ki67 in epidermis: 0-None; 1-Single cell layer IR at basal zone of epidermis; 2-IR 2-5 layers thick within the basal zone of epidermis; 3-IR>5 cell layers thick within the basal zone of epidermis. The data is presented as a mean±S.E.M. (n=5). The statistical analysis was performed using ordinary one-way ANOVA with an uncorrected Fisher's LSD test for a multiple comparison, *p<0.05, **p<0.005.

FIGS. 5A and 5B show that NTCI treatment reduces PMA-Induced ear inflammation. A single dose of Phorbol Myristate Acetate (PMA, 2 nmoles in 10 μl EtOH on each side) was administered topically to the right ear of 8-week-old female C57Bl/6 mice. Topical treatment with NTCI, cSN50.1 peptide (16.7 nmoles in 10 μl EtOH on each side), or vehicle (10 μl EtOH on each side) was administered 30 min before and 3, 6, and 8 h. after PMA challenge. A control group of mice was treated topically with vehicle only (10 μl EtOH) following the same treatment schedule. FIG. 5A shows ear thickness was measured at 30 min before (0 h) and 3, 6, 8 and 24 h. after PMA challenge. Measurements are represented as mean±S.E.M. (n=5). Statistical significance of the difference between ear thickness in NTCI-treated mice (PMA+cSN50.1) and vehicle treated (PMA+Vehicle) or vehicle control (Vehicle) groups was determined by two-way ANOVA using corrected Holm-Sidak test for multiple comparison, ***p<0.0005. FIG. 5B shows ear images show increased swelling and redness induced by PMA (PMA+Vehicle). H & E staining of paraffin-embedded ear punch biopsies, collected 8 h. after PMA challenge, show increased swelling. Both signs of inflammation (redness and swelling) are reduced by NTCI treatment (PMA+cSN50.1). Displayed are representative images (×20 magnification) of pinna sections. The NTCI suppresses Mrgpra2a, the mouse homolog to the human gene encoding MRGPRX2, the key Mas-related G protein-coupled receptor-X2 mediating mast-cell activation in response to a broad array of insults. They include: wasp venom and several pharmaceutical compounds associated with the IgE-independent pseudo-allergic reactions in patients.

FIG. 6 shows the mechanism of NTCI action in AD through targeted inhibition of importins α5 and β1 comprising the nuclear transport checkpoint. Proinflammatory Stress-Responsive Transcription Factors, which are activated by allergic, autoimmune, and microbial inducers of inflammation are ferried to the cell's nucleus through an Importin α5-mediated pathway. Metabolic Transcription Factors, which are activated by overfeeding or inborn errors of metabolism are shuttled to the nucleus by Importin β1. NTCI stops binding of Transcription Factors to Importin α5 and Importin β1 thereby preventing the activation of the genes encoding mediators of Atopic Dermatitis as well as genes encoding metabolic intermediates. The latter underlie the link between AD and Metabolic Syndrome (see text and FIG. 7 for expanded view of proinflammatory signaling to the nucleus comparing NTCI action with currently used drugs in AD). NPC, Nuclear Pore Complex.

FIG. 7 shows the inducers of Allergic, Autoimmune, Microbial, and Metabolic Inflammation Trigger Signaling Pathways that Mobilize Eight Families of Transcription Factors to Reach the Cell's Nucleus and Activate Multiple Genes Encoding Mediators of Inflammation. Nuclear Transport Checkpoint Inhibitor (NTCI) targets Importin α5 and Importin β1, which shuttle eight depicted transcription factor families thereby arresting their passage through the Nuclear Pore Complex (NPC). Hence, multiple inflammatory genes, that encode mediators of allergic, autoimmune, microbial, and metabolic inflammation, are suppressed by NTCI. For comparison, the currently used anti-inflammatory drugs for treatment of Atopic Dermatitis target single inflammatory mediators depicted in black rectangles. Glucocorticoids are not displayed (see text for their mechanism of action).

FIGS. 8A, 8B, 8C, and 8D show that NTCI treatment of stimulated human KCs inhibits proinflammatory signaling to the nucleus by reducing the translocation of NF-κB RelA and STAT3 and inhibits the expression of TSLP and other proinflammatory genes involved in the inflammation of the skin. A. The human primary keratinocytes derived from adult skin were pretreated with NTCI (cSN50.1 peptide) 30 minutes before stimulation with a mix of two cytokines: TNF-α and IL-17. The nuclear content of the SRTFs, NF-κB RelA and pSTAT3 (phosphorylated STAT3), was determined by a quantitative immunoblotting analysis using the Li-Cor Odyssey Infrared Imaging System. The data is presented as a mean±S.E.M. (n=4). The statistical significance of a difference between the nuclear content of SRTFs in the TNF-α/IL-17-stimulated cells (Stimulated) and stimulated cells treated with the NTCI peptide cSN50.1 (Stim.+cSN50.1) was determined by an ordinary one-way ANOVA, **p<0.005 (the unedited full-length immunoblots are presented in the FIG. 10). B. The expression of the genes encoding the mediators of inflammation was determined using real-time quantitative reverse transcription PCR (qRT PCR). The relative expression levels were established with Livak's methods (2^{-ΔΔCt}) with αTubulin gene as the reference and a Mock Control group as the calibrator. The data is presented as a mean±S.E.M. (n=4). The statistical analysis was performed using an ordinary one-way ANOVA, **p<0.005, ***p<0.0005. C. Suppression of TSLP, GM-CSF (CSF2), and IL-8 (CXCL8) production in cultured human keratinocytes stimulated with TNF-α/IL-17. Supernatant samples were collected from the medium of cultured keratinocytes before (0 h) and 24 and 48 hours after TNF-α/IL-17 stimulation. The proteins' concentration in the culture medium was determined using ELISA (TSLP) and CBA (GM-CSF and IL-8) assays. The data is presented as mean±S.E.M. (n=4). The statistical significance of the difference between the cytokine/chemokine levels in the media from stimulated cells and stimulated cells treated with the NTCI peptide cSN50.1 was determined by a repeated measure two-way ANOVA, ***p<0.0005, ****p<0.0001. D. Suppression of TSLP, GM-CSF (CSF2), IL-8 (CXCL8), TNF-α, and IL-6 production in cultured human keratinocytes stimulated with PMA. The human primary keratinocytes derived from adult skin were pretreated with NTCI (cSN50.1 peptide) 30 minutes before stimulation with PMA. Supernatant samples were collected from the medium of cultured keratinocytes before (0 h) and 6 and 24 hours after PMA challenge. The proteins concentration in the culture medium were determined using ELISA (TSLP) and CBA (TNF-, α IL-6, GM-CSF and IL-8) assays. The data is presented as mean #S.E.M. (n=4). The statistical significance of the difference between the cytokine/chemokine levels in the media from stimulated cells and stimulated cells treated with the NTCI peptide cSN50.1 was determined by a repeated measure two-way ANOVA, ****p<0.0001.

FIG. 9 shows the mechanism of the NTCI cytoprotective action in inflamed human primary keratinocytes. A mix of cytokines TNF-α and IL-17 or skin irritant, PMA, activate the proinflammatory signaling pathways. These pathways are transduced by two SRTFs, NF-κB/RelA and pSTAT3 known to activate proinflammatory and proapoptotic genes. The nuclear translocation of NF-κB/RelA and pSTAT3 is mediated by a nuclear import adaptor complex, Imp α5/Imp β1. The NTCI (cell-penetrating cSN50.1 peptide) controls this checkpoint. Consequently, selective signaling to the nucleus by SRTFs is stopped and the activation of genes encoding the mediators of inflammation and apoptosis is suppressed [see FIG. 10 in reference for more details of this and other signaling pathways as well as the points of action of other anti-inflammatory therapeutics). NPC, Nuclear Pore Complex.

FIG. 10 shows unedited full-length immunoblots used for preparation of FIG. 8A. Immunoblot analysis of NF-κB/RelA and pSTAT3 in nuclear fractions of primary human keratinocytes stimulated with TNF-α and IL-17. SDS PAGE electrophoreses were performed on 10% gels. Protein samples were transferred into cellulose membranes using Trans-Blot Turbo System (Bio-Rad). Membranes were immunoblotted with rabbit polyclonal anti-Histone 3 primary antibody (Cell Signaling Technology, #9715) and rabbit monoclonal anti-NF-κB p65 (RelA) antibody (Cell Signaling Technology, #8242) following blotting with goat anti-rabbit IRDye 680RD secondary antibodies for detection in 700 nm channel. After immunoblotting was completed, membranes were scanned with 700 nm channel on Odyssey CLx Infrared Imaging System (LI-Cor). The membrane was then reblotted with rabbit polyclonal anti p-STAT3 (Cell Signaling Technology, #9131) following blotting with goat anti-rabbit IRDye 680RD secondary antibodies. The membrane was then rescanned using the Odyssey CLx Infrared Imaging System. Images were quantitatively analyzed using Image Studio 3.1 software (Li-Cor).

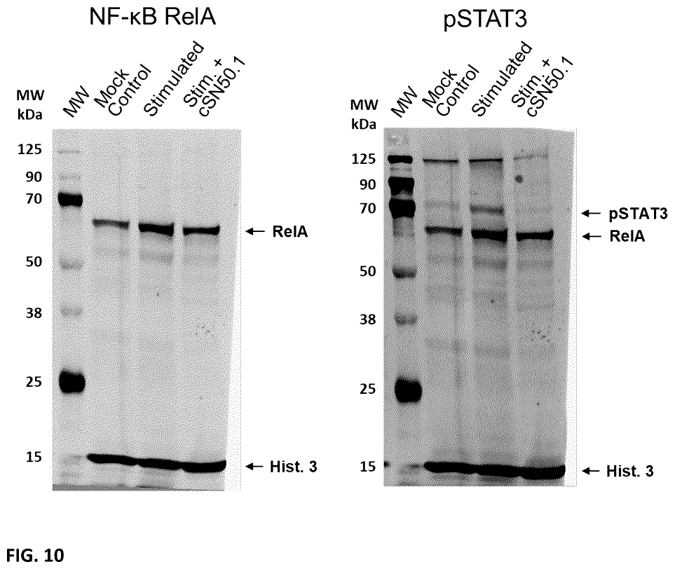

### VII. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Using Nuclear Transport Checkpoint Inhibitors (NTCI)

Although compositions, kits, cells, and methods similar or equivalent to those described herein can be used in the practice or testing of the present invention, suitable compositions, kits, cells, and methods are described below. All publications, patent applications, and patents mentioned herein are incorporated by reference in their entirety. U.S. patents applications Ser. Nos. 14/349,918, and U.S. Pat. No. 7,553,929, for example, are incorporated by reference in their entireties. In the case of conflict, the present specification, including definitions, will control. The particular embodiments discussed below are illustrative only and not intended to be limiting.

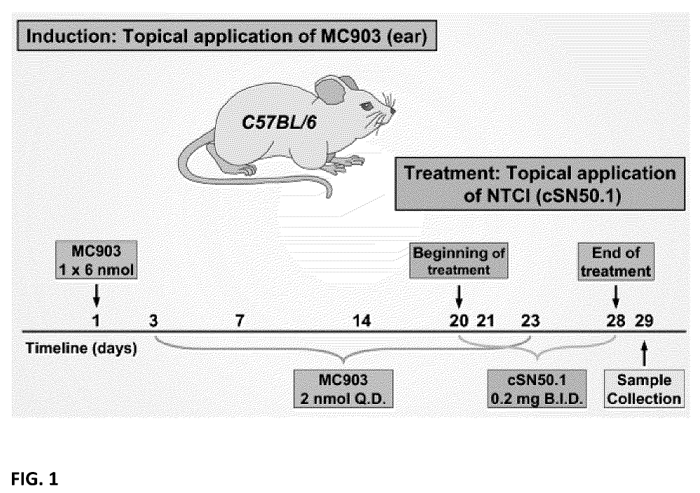

Small transcription factors (<45 kD), usually those regulating the housekeeping genes that encode cell survival factors, have free passage from the cytoplasm to the nucleus. In contrast, nuclear transport of transcription factors larger than 45 kD, such as SRTFs, is guided by one or more nuclear localization sequences (NLSs). These intracellular “zip codes” are displayed on SRTFs upon stimulation of immune and non-immune cells by microbial insults. NLSs are then recognized by nuclear transport adaptor proteins, importins/karyopherins alpha (Imp α) (see FIG. 1A). The stimulus-induced formation of SRTF and importins α complexes also encompasses importin beta 1 (Imp β1), which is recognized by nuclear pore proteins to allow translocation of the cargo to the nucleus. Until recently, nuclear transport has been targeted through the forced expression of genes that encode inhibitors of proinflammatory SRTFs, such as the degradation-resistant inhibitor of NF-κB termed IκBα. However, NF-κB is only one of multiple SRTFs that mediate signaling to the nucleus in response to infection. Other SRTFs, such as AP-1, STAT1 and NFAT, are also transported to the nucleus during the inflammatory response yet their nuclear transport is not impeded by IκBα; contrarily, the AP-1 pathway is activated. Targeting nuclear transport, a pivotal checkpoint integrating translocation of multiple transcription factors to the nucleus, can be a more efficient strategy than targeting signaling pathways of individual transcription factors. This concept was proven by design and development of NTCIs.

NTCIs target the nuclear transport shuttles, Imp α5 and Imp β1, that translocate SRTFs to the nucleus and control signal transduction pathways, which culminate in genomic reprogramming. NTCIs modulate signaling to the nucleus mediated by transcription factors that include but are not limited to NFκB, AP-1, NFAT, STAT1 that utilize importins alpha and beta heterodimer, or SREBP1a, SREBP1c, and SREBP2, that utilize solely importin beta for nuclear transport whereas ChREBP can utilize primarily importins alpha/beta heterodimer for nuclear translocation. SRTFs such as NFκB, AP-1, NFAT, STAT1 are transported to the nucleus in response to proinflammatory stimuli. In the nucleus, SRTFs activate genes that encode mediators of inflammation. Examples of NTMs include SN50, cSN50 and cSN50.1 described in more detail in the following paragraphs, as well as the sequences set forth in Table 2.

In recent preclinical studies, a highly soluble cell-penetrating NTCI aka NTM (cSN50.1), with dual specificity was used. This NTCI has segments that bind both Imp α5, which recognizes NLS derived from NFκB1, and Imp β1, which recognizes the signal-sequence hydrophobic region (SSHR) derived from Fibroblast Growth Factor 4. SSHR also serves as a membrane translocating motif (MTM) to enable intracellular delivery of peptides and proteins through an ATP-and endocytosis-independent mechanism. This and other NTCIs have been shown to inhibit nuclear translocation of SRTFs and metabolic transcription factors, Sterol Regulatory Element Binding Proteins (SREBPs) thereby reducing inflammatory responses, microvascular injury, apoptosis and hemorrhagic necrosis as well as correcting metabolic derangements (eg. hyperlipidemia, with a concomitant gain in survival, in models of lethal shock induced by bacterial toxins.

A novel form of immunotherapy that targets nuclear import, as described herein, can arrest inflammation-driven destruction of microbe-infected tissue and surrounding area of a given organ. With respect to microbial inflammation (such as, for example, acute inflammation, subacute inflammation, chronic inflammation, skin-specific inflammation, systemic inflammation), pro-inflammatory signaling initiated through stimulation of the principal receptors of innate immunity, Toll-like receptors (TLRs), is one mechanism that activates antigen-presenting cells (APCs). Reprograming of gene regulatory networks in response to a multitude of microbial insults is dependent on signaling to the host cell's nucleus comprising a fundamental process of microbial inflammation (see FIG. 1A for a depiction). Inhibiting nuclear transport at a common “checkpoint” located downstream of TLRs and cytokine receptors globally suppresses expression of inflammatory genes thereby calming the genomic storm and averting multiple organ injury.

Accordingly, in one aspect, disclosed herein are methods of diagnosing an inflammatory skin disease or inflammatory skin disorder caused by a microbial, allergic, autoimmune, constitutive, metabolic, neoplastic and/or physical insults (such as, for example, atopic dermatitis) in a subject the methods comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates the subject has a skin disease or inflammatory skin disorder.

It is understood and herein contemplated that the ability to diagnose an inflammatory skin disease or inflammatory skin disorder caused by a microbial, allergic, autoimmune, constitutive, metabolic, neoplastic and/or physical insults (such as, for example, atopic dermatitis) can lead directly to an incorporated in a method of treating/inhibiting/reducing said inflammatory skin disease or inflammatory skin disorder caused by a microbial, allergic, autoimmune, constitutive, metabolic, neoplastic and/or physical insults (such as, for example, atopic dermatitis). Thus, in one aspect, disclosed herein are methods of treating/inhibiting/reducing an inflammatory skin disease or inflammatory skin disorder caused by a microbial, allergic, autoimmune, constitutive, metabolic, neoplastic and/or physical insults (such as, for example, atopic dermatitis) in a subject, the method comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to the subject with elevated expression or activity of one or more of TSLP, IL-1β, IL-4, IL-4Rα, IL-6, IL-8, IL-10, IL-13, IL-17, IL-18, IL-23a, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111, a therapeutically effective amount of a composition comprising a Nuclear Transport Checkpoint Inhibitors (NTCIs), also known as Nuclear Transport Modifiers (NTMs).

The disclosed NTCIs can reduce, inhibit, and/or prevent skin inflammation (such as, for example, a skin disorder caused by microbial agents that induce microbial inflammation, autoimmune disorder, alloimmune disorder, allergens (i.e., allergic process), autoinflammatory disorder, metabolic process, neoplastic/oncogenic disorder, or physical insults that are mediated by inflammation (such as, for example, atopic dermatitis) that are mediated by inflammation, as displayed in Table 1, and including, but not limited to contact dermatitis, psoriasis, systemic lupus erythematosus, bullous dermatitis, “flesh-eating disease”, seborrheic dermatitis, atopic dermatitis, and graft-versus-host disease). Accordingly, described herein is a method of treating, inhibiting, reducing, and/or preventing skin diseases (such as, for example, mediated by microbial inflammation, autoimmune inflammation, allergic inflammation, metabolic inflammation, neoplastic inflammation, and physical inflammation as exemplified in Table 1 comprising administering to the subject with the skin disease mediated by microbial inflammation a composition comprising NTM in combination with one or more anti-microbial agents.

TABLE 1
51. Skin Diseases Mediated by Inflammation

| Type of | Cause of | Examples of Skin Diseases Mediated |
|---|---|---|
| Inflammation | Inflammation | by a Given Type of Inflammation |
| Microbial | Bacteria, | Abscess, Acne, Blepharitis, Candidiasis, |
| Inflammation | Fungi, Mites, | Dermatophytic Infections of the Folds, |
| | and Viruses | Erysipelas, Fistulas, Furunculosis, |
| | | Genital Warts, Impetigo, Seborrheic |
| | | Dermatitis*, Moluscum Contagiosum, |
| | | Necrotizing Fasciitis, Paronychia, |
| | | Purpura Fulminans, Scabies, |
| | | Shingles, Tinea Faciei |
| Autoimmune | Aberrant | Erythema Nodosum, Fistulas and Oral |
| Inflammation | Autoimmune | Ulcers in Crohn Disease, Graft-Versus- |
| | Attack | Host Disease, Pemphigus, Psoriasis, |
| | | Scleroderma, Systemic Lupus |
| | | Erythematosus, the IgE-independent |
| | | pseudo-allergic reactions |
| Allergic | Allergens | Atopic Dermatitis/Eczema, Contact |
| Inflammation | | Dermatitis including Poison Ivy, Oak, |
| | | and Sumac, Drug Hypersensitivity |
| | | Reactions, Insect Bites |
| Constitutive | Inborn Errors | Familial Mediterranean Fever, NEMO |
| Inflammation | of Innate | Mutation-Linked Intestinal and Skin |
| | Immunity | Autoinflammatory Disease |
| Metabolic | Excessive | Seborrheic Dermatitis*, Gout (Tophi), |
| Inflammation | Accumulation | Xanthelasma |
| | of Metabolites | |
| | (e.g. sebum, | |
| | cholesteryl | |
| | esters or uric | |
| | acid deposits) | |
| Physical | Trauma, | Bedsores, Chemical, Electric and |
| Inflammation | Burns, or | Thermal (scalding) Burns, |
| | Radiation | Radiation Injury, Skin Aging |
| Neoplastic | Unknown | Mycosis Fungoides, Sezary Syndrome |
| Inflammation | Kaposi's | Kaposi's Sarcoma |
| | Sarcoma- | |
| | Associated | |
| | Herpesvirus | |
| | Human | Adult T cell Leukemia/Lymphoma |
| | T-Cell | with skin erythematous and itchy |
| | Leukemia | plaques |
| | Virus-1 | |

*Seborrheic Dermatitis is mediated by both Metabolic and Microbial Inflammation

In one aspect, the methods treating, inhibiting, reducing, and/or preventing skin inflammation includes administering a therapeutically effective amount of a composition comprising one or more NTM to the mammalian subject. Administration of the composition decreases inflammation by attenuating expression of at least one stress-responsive transcription factor-regulated gene, or at least one ChREBPs and/or one SREBPs-regulated gene. Thus, the effective dose is an amount effective for reducing importin alpha-mediated nuclear translocation of at least one stress response SRTF or one ChREBPs and reducing skin inflammation (such as, for example, a skin disorder caused by microbial agents that induce microbial inflammation, allergen (i.e., allergic process), autoimmune disorder, alloimmune disorder, autoinflammatory disorder, metabolic process, neoplastic disorder, and/or physical factors and/or insults that are mediated by inflammation, including, but not limited to contact dermatitis, psoriasis, systemic lupus erythematosus, bullous dermatitis, necrotizing fasciitis aka “flesh-eating disease”, seborrheic dermatitis, atopic dermatitis, and graft-versus-host disease) in the mammalian subject. Similarly, the effective dose is an amount effective for reducing importin beta-mediated nuclear translocation of at least one metabolic transcription factors, SREBP and reducing a skin inflammation in the mammalian subject. The NTM may bind to importin alpha, to importin beta, or to both importin alpha and importin beta.

An important aspect of the NTCI exemplified by the cSN50.1 peptide and its congeners is their ability to reach the site of infection and the infected host cell in the skin, as well as other myeloid, lymphoid, and non-lymphoid organs. The mechanism of intracellular delivery of this class of cell-penetrating peptides has been elucidated and an endocytosis-independent process of crossing the plasma membrane mediated by the membrane-translocating motif (MTM), which is based on the SSHR derived from Kaposi FGF, has been documented (Veach et al. (2004) J Biol Chem 279:11425-11431). The amphipathic helix-based structure of SSHR facilitates its insertion directly into the plasma membrane and the tilted transmembrane orientation permits the translocation of the NTM through the phospholipid bilayer of the plasma membrane directly to the interior of the cell without perturbing membrane integrity. This mechanism explains the efficient delivery of SSHR-guided cargo across the plasma membrane of multiple cell types involved in microbial inflammation, autoimmune inflammation, allergic inflammation, metabolic inflammation, neoplastic inflammation, and physical inflammation that mediate skin diseases.

The NTMs disclosed herein are derived from N50-containing NTMs (SN50, cSN50, and cSN50.1) that are comprised of a hydrophilic N50 motif patterned on the nuclear localization sequence (NLS) region of the NFκB1/p50 subunit (see Table 2) fused to a motif from the signal SSHR of human fibroblast growth factor 4. The SSHR allows peptides to cross the plasma membrane by an ATP- and endosome-independent mechanism, and the N50 motif was designed to bind to importins α during stimulus-initiated signaling and thereby limit docking of NLS-bearing SRTFs to their adaptor proteins and reduce nuclear import of activated STRFs. Any mimetics, derivatives, or homologs of SN50, cSN50, and cSN50.1 may be used in the compositions, methods, and kits disclosed herein.

TABLE 2
Amino Acid Sequences of Peptides Used
Peptide Sequences

| NTM | SSHR | NLS | SEQ ID NO: |
|---|---|---|---|
| N50 | | VQRKRQKLMP | 10 |
| N50M | | VQRDEQKLMP | 11 |
| cN50.1 | | CVQRKRQKLMPC | 12 |
| SN50 | AAVALLPAVLLALLAP | VQRKRQKLMP | 13 |
| SSHR-1 | AAVALLP | | 14 |
| SSHR-2 | AVLLALLAP | | 15 |

N50-sequence derived from the NLS region of NFκB1/p50; N50M-sequence of control peptide with KR to DE mutation (bolded); cN50.1-sequence of cyclized version of N50 just as cSN50.1 is a cyclized version of SN50.
Hydrophobic regions of the SSHR domain are distinguished from the cluster of basic amino acids (NLS).
NTM indicates nuclear transport modifier; SSHR, signal sequence hydrophobic region; NLS, nuclear localization sequence.

SN50 is a fragment linked peptide combining the SSHR of the Kaposi fibroblast growth factor 4 (K-FGF4) and the NLS of the p50 subunit of NFκB1. Any mimetics, derivatives, or homologs of SN50 may be used in the compositions, methods, and kits disclosed herein. The sequence of SN50 is AAVALLPAVLLALLAPVQRKROKLMP (SEQ ID NO: 13). Generation and use of SN50 is described in U.S. Pat. No. 7,553,929.

cSN50 is a fragment-designed cyclic peptide combining the hydrophobic region of the Kaposi fibroblast growth factor signal sequence with the nuclear localization signal (NLS) of the p50-NFκB1 and inserting a cysteine on each side of the NLS to form an intrachain disulfide bond. The amino acid sequence of cSN50 is AAVALLPAVLLALLAPCYVQRKRQKLMPC (SEQ ID NO: 1). Any mimetics, derivatives, or homologs of cSN50 may be used in the compositions, methods, and kits disclosed herein. Methods of making and using cSN50 are described, for example, in U.S. Pat. Nos. 7,553,929 and 6,495,518. These patents are incorporated herein by reference in their entireties.

cSN50.1 is a cyclized peptide having the sequence of cSN50 with the exception that the tyrosine at position 18 of cSN50, adjacent to the first cysteine, has been removed. Methods of making and using cSN50 are described, for example, in U.S. Pat. Nos. 7,553,929 and 6,495,518. The amino acid sequence of cSN50.1 is AAVALLPAVLLALLAPCVQRKRQKLMPC (SEQ ID NO: 2). The tyrosine at position 18 was removed from the sequence of cSN50 to increase solubility. cSN50 is soluble at levels of ranging from 2.0 mg/mL to 40 mg/mL depending on the method of synthesis and purification whereas cSN50.1 is soluble at levels of at least 100 mg/ml. Any mimetics, derivatives, or homologs of cSN50.1 may be used in the compositions, methods, and kits disclosed herein. cSN50.1 is also encompassed by SEQ ID NO: 3, SEQ ID NO: 4 and SEQ ID NO: 5. Additional examples of NTMs include fragment-designed and synthesized peptides in which cargo is incorporated as two, rather than one, modules or cargos derived from intracellular proteins other than NFκB 1. Such additional examples include the sequences of SEQ ID NO: 6, SEQ ID NO: 7, SEQ ID NO: 8, and SEQ ID NO: 9.

Accordingly, the NTM for use in the disclosed methods of treating, inhibiting, reducing, and/or preventing inflammatory skin diseases including, but not limited to microbial disease, autoimmune disease, autoinflammatory disease, metabolic disorder, neoplastic disorder, or physical injuries that are mediated by inflammation may be, for example, an NTM having the sequence Xaa Xaa Xaa Xaa Leu Leu Pro Xaa Xaa Leu Leu Ala Leu Leu Ala Pro Xaa Xaa Xaa Gln Arg Lys Arg Gln Lys Xaa Xaa Xaa Xaa (SEQ ID NO: 3), wherein Xaa is any amino acid or is absent. For example, the Nuclear Transport Modifier can have the sequence Xaa Xaa Xaa Xaa Leu Leu Pro Xaa Xaa Leu Leu Ala Leu Leu Ala Pro Cys Xaa Xaa Gln Arg Lys Arg Gln Lys Xaa Xaa Xaa Cys, where Xaa is any amino acid or is absent (SEQ ID NO: 4). As another example, the Nuclear Transport Modifier can have the sequence Xaa Xaa Xaa Xaa Leu Leu Pro Xaa Xaa Leu Leu Ala Leu Leu Ala Pro Cys Xaa Gln Arg Lys Arg Gln Lys Xaa Xaa Xaa Cys, where Xaa is any amino acid or is absent (SEQ ID NO: 5). In one embodiment, the Nuclear Transport Modifier is cSN50.1 having the sequence set forth in SEQ ID NO: 2. In another example of an NTM, the NTM has the sequence Xaa Xaa Xaa Xaa Leu Leu Pro Xaa Xaa Leu Leu Ala Val Leu Ala Pro Xaa Xaa Xaa Gln Arg Lys Arg Gln Lys Xaa Xaa Xaa Xaa, where Xaa is any amino acid or is absent (SEQ ID NO: 6). In yet another example, the NTM has the sequence Ala Ala Val Ala Leu Leu Pro Ala Val Leu Leu Ala Val Leu Ala Pro Cys Val Gln Arg Lys Arg Gln Lys Leu Met Pro Cys (SEQ ID NO: 7). In a further example, the NTM has the sequence Xaa Xaa Xaa Xaa Leu Leu Pro Xaa Xaa Leu Leu Ala Val Leu Ala Pro Xaa Xaa Xaa Gln Arg Asp Glu Gln Lys Xaa Xaa Xaa Xaa, where Xaa is any amino acid or is absent (SEQ ID NO: 8). In another example, the NTM has the sequence Ala Ala Val Ala Leu Leu Pro Ala Val Leu Leu Ala Val Leu Ala Pro Cys Val Gln Arg Asp Glu Gln Lys Leu Met Pro Cys (SEQ ID NO: 9).

1. Compositions for Treating Skin Diseases and Disorders Associated with Inflammation in a Subject

Compositions (e.g., pharmaceutical compositions) described herein for treating diseases associated with inflammation include a pharmaceutically acceptable carrier and at least one importin beta-selective and/or at least one importin alpha-selective NTCI aka NTM in an amount effective for modifying (e.g., decreasing) entry into the nucleus of at least one transcription factor that includes but is not limited to NFκB, AP-1, NFAT, STAT1, ChREBPα, ChREBPβ, SREBP1a, SREBP1c, and SREBP2, that utilize importins alpha and/or beta for nuclear transport, and treating or preventing the disease. For example, entry of at least one SREBP into the nucleus is reduced. As mentioned above, NTMs modulate signaling to the nucleus mediated by transcription factors that include but are not limited to NFκB, AP-1, NFAT, STAT1 that utilize importins alpha and beta heterodimer, SREBP1a, SREBP1c, and SREBP2, that utilize solely importin beta for nuclear transport whereas ChREBP can utilize primarily importins alpha for nuclear translocation. In this example, the importin beta-selective NTM reduces nuclear translocation of the nuclear forms of SREBP1a, SREBP1c, and SREBP2. Any suitable importin beta-selective NTM may be used. Examples of importin beta-selective NTMs include but are not limited to peptide sequences that include an SSHR domain listed in Table 3 below and a cargo listed in Table 2 above. One example of such an importin beta-selective NTM is AAVALLPAVLLALLAPVQRDEQKLMP (SEQ ID NO: 40) (i.e., a peptide sequence having the SSHR domain of AAVALLPAVLLALLAP (SEQ ID NO: 17) and the cargo of VQRDEQKLMP (SEQ ID NO: 11) as listed in Table 3 below). Additional examples of peptides designed to inhibit interaction of importin alpha with importin beta necessary for the formation of their heterodimer include AAVALLPAVLLALLAPRRRRIEVNVELRKAKK (SEQ ID NO: 18) (referred to as SIBB in Table 3), AAVALLPAVLLALLAPRRRRIEVNVELRKAKKDD (SEQ ID NO: 19) (referred to as SI-1 in Table 3). AAVALLPAVLLALLAPRRQRNEVVVELRKNKRDE (SEQ ID NO: 20) (referred to as SI-3 in Table 3), AAVALLPAVLLALLAPRRHRNEVTVELRKNKRDE (SEQ ID NO: 21) (referred to as SI-4 in Table 3), AAVALLPAVLLALLAPRRRREEEGLQLRKQKREE (SEQ ID NO: 22) (referred to as SI-5 in Table 3), AAVALLPAVLLALLAPRRRREEEGIQLRKQKREQ (SEQ ID NO: 23) (referred to as SI-7 in Table 3) and AAVALLPAVLLALLAPCTEMRRRRIEVC (SEQ ID NO: 24) (referred to as cSIB in Table 3). The examples of peptides designed to be specific inhibitors of importins alpha include AAVALLPAVLLALLAPVELRKAKKDDQMLKRRNVSSF (SEQ ID NO: 25) (referred to as SAR1 in Table 3), AAVALLPAVLLALLAPVELRKNKRDEHLLKRRNVPHE (SEQ ID NO: 26) (referred to as SAR3 in Table 3), AAVALLPAVLLALLAPVELRKNKRDEHLLKKRNVPQE (SEQ ID NO: 27) (referred to as SAR4 in Table 3), AAVALLPAVLLALLAPLQLRKQKREEQLFKRRNVATA (SEQ ID NO: 28) (referred to as SAR5 in Table 3), AAVALLPAVLLALLAPIQLRKQKREQQLFKRRNVELI (SEQ ID NO: 29) (referred to as SAR7 in Table 3), AAVALLPAVLLALLAPCVELRKAKKDDQC (SEQ ID NO: 30) (referred to as cSAR1-C in Table 3), AAVALLPAVLLALLAPCVELRKNKRDEHC (SEQ ID NO: 31) (referred to as cSAR3-C in Table 3), AAVALLPAVLLALLAPCLQLRKQKREEQC (SEQ ID NO: 32) (referred to as cSAR5-C in Table 3), AAVALLPAVLLALLAPCIQLRKQKREQQC (SEQ ID NO: 33) (referred to as cSAR7-C in Table 3), AAVALLPAVLLALLAPCQMLKRRNVSSFC (SEQ ID NO: 34) (referred to as cSAR1-N in Table 3), AAVALLPAVLLALLAPCHLLKRRNVPHEC (SEQ ID NO: 35) (referred to as cSAR3-N in Table 3), AAVALLPAVLLALLAPCHLLKKRNVPQEC (SEQ ID NO: 36) (referred to as cSAR4-N in Table 3), AAVALLPAVLLALLAPCQLFKRRNVATAC (SEQ ID NO: 37) (referred to as cSAR5-N in Table 3), and AAVALLPAVLLALLAPCQLFKRRNVELIC (SEQ ID NO: 38) (referred to as cSAR7-N in Table 3). It is to be understood that any derivatives and/or analogues of these sequences are encompassed by the invention.

In one embodiment, an NTM as described herein has the sequence AAVALLPAVXLAXXAPVELRKNKRDEHLLKRRNVPHE (SEQ ID NO: 39). Additional NTMs include SEQ ID NOs: 1-9, 13, and 16-41. It is to be understood that any derivatives and/or analogues of these sequences are encompassed by the invention.

An NTM as described herein may be an inhibitor of an importin alpha 3 interaction with importin beta.

The SI-3 sequence (see Table 3) is designed to block an interaction between importin alpha and importin beta. Hence, this peptide is a cell-penetrating inhibitor of an importin alpha and importin beta interaction. It is to be understood that any derivatives and/or analogues of this sequence is encompassed by the invention.

TABLE 3
Peptide sequences

| | | | SEQ | |
|---|---|---|---|---|
| | | | ID | |
| | SSHR^{§} | Cargo | NO: | Comments |
| SM12 | AAVALLPAVLLALLAP | VQRDEQKLMP | 40 | Importin beta- |
| | | | | selective inhibitor |
| | | | | (binding studies) |
| SIBB | AAVALLPAVLLALLAP | RRRRIEVNVELRKAKK | 18 | Inhibitor of Imp |
| | | | | alpha 1-importin |
| | | | | beta interaction |
| SI-1 | AAVALLPAVLLALLAP | RRRRIEVNVELRKAKK | 19 | Inhibitor of Imp |
| | | DD | | alpha 1-importin |
| | | | | beta interaction |
| SI-3 | AAVALLPAVLLALLAP | RRQRNEVVVELRKNK | 20 | Inhibitor of Imp |
| | | RDE | | alpha 3-importin |
| | | | | beta interaction |
| SI-4 | AAVALLPAVLLALLAP | RRHRNEVTVELRKNK | 21 | Inhibitor of Imp |
| | | RDE | | alpha 4-importin |
| | | | | beta interaction |
| SI-5 | AAVALLPAVLLALLAP | RRRREEEGLQLRKQKR | 22 | Inhibitor of Imp |
| | | EE | | alpha 5-importin |
| | | | | beta interaction |
| SI-7 | AAVALLPAVLLALLAP | RRRREEEGIQLRKQKR | 23 | Inhibitor of Imp |
| | | EQ | | alpha 7-importin |
| | | | | beta interaction |
| SAR1 | AAVALLPAVLLALLAP | VELRKAKKDDQMLKR | 25 | Imp alpha 1- |
| | | RNVSSF | | specific |
| SAR3 | AAVALLPAVLLALLAP | VELRKNKRDEHLLKR | 26 | Imp alpha 3- |
| | | RNVPHE | | specific |
| SAR4 | AAVALLPAVLLALLAP | VELRKNKRDEHLLKK | 27 | Imp alpha 4- |
| | | RNVPQE | | specific |
| SAR5 | AAVALLPAVLLALLAP | LQLRKQKREEQLFKRR | 28 | Imp alpha 5- |
| | | NVATA | | specific |
| SAR7 | AAVALLPAVLLALLAP | IQLRKQKREQQLFKRR | 29 | Imp alpha 7- |
| | | NVELI | | specific |
| cMN50.1 | AAVALLPAVXLAXXAP | CVQRKRQKLMPC | 17 | Imp alpha 5- |
| | | | | selective |
| cSN50.1β | AAVALLPAVLLALLAP | CVQRDEQKLMPC | 16 | Imp beta-selective |
| | | | | (cell culture and |
| | | | | preclinical studies) |
| cSIB | AAVALLPAVLLALLAP | CTEMRRRRIEVC | 24 | Inhibitor of Imp |
| | | | | alpha 1-importin |
| | | | | beta interaction |
| cSAR1-C | AAVALLPAVLLALLAP | CVELRKAKKDDQC | 30 | Imp alpha 1- |
| | | | | specific |
| | | | | Proximal to C- |
| | | | | terminal |
| cSAR3-C | AAVALLPAVLLALLAP | CVELRKNKRDEHC | 31 | Imp alpha 3- |
| | | | | specific |
| | | | | Proximal to C- |
| | | | | terminal |
| cSAR5-C | AAVALLPAVLLALLAP | CLQLRKQKREEQC | 32 | Imp alpha 5- |
| | | | | specific |
| | | | | Proximal to C- |
| | | | | terminal |
| cSAR7-C | AAVALLPAVLLALLAP | CIQLRKQKREQQC | 33 | Imp alpha 7- |
| | | | | specific |
| | | | | Proximal to C- |
| | | | | terminal |
| cSAR1-N | AAVALLPAVLLALLAP | CQMLKRRNVSSFC | 34 | Imp alpha 1- |
| | | | | specific |
| | | | | Proximal to N- |
| | | | | terminal |
| cSAR3-N | AAVALLPAVLLALLAP | CHLLKRRNVPHEC | 35 | Imp alpha 3- |
| | | | | specific |
| | | | | Proximal to N- |
| | | | | terminal |
| cSAR4-N | AAVALLPAVLLALLAP | CHLLKKRNVPQEC | 36 | Imp alpha 4- |
| | | | | specific |
| | | | | Proximal to N- |
| | | | | terminal |
| cSAR5-N | AAVALLPAVLLALLAP | CQLFKRRNVATAC | 37 | Imp alpha 5- |
| | | | | specific |
| | | | | Proximal to N- |
| | | | | terminal |
| cSAR7-N | AAVALLPAVLLALLAP | CQLFKRRNVELIC | 38 | Imp alpha 7- |
| | | | | specific |
| | | | | Proximal to N- |
| | | | | terminal |

^{§}Signal Sequence Hydrophobic Region (SSHR)
“Cargo” comprises sequences of functionally active hydrophilic motifs (fragments) listed as linear or cyclized peptides through addition of cysteine at the amino- and carboxy-termini of respective linear peptides. Both linear and cyclized sequences are fused to hydrophobic membrane translocation motif denoted SSHR.

In one aspect disclosed herein are methods of treating, inhibiting, reducing, and/or preventing inflammatory skin disorder (such as, for example, a skin disorder caused by microbial agents that induce microbial inflammation, autoimmune disorder, alloimmune disorder, autoinflammatory disorder, allergen (i.e., allergic process), metabolic process, neoplastic disorder, and/or physical factors and/or insults that are mediated by inflammation, including, but not limited to contact dermatitis, psoriasis, systemic lupus erythematosus, bullous dermatitis, necrotizing fasciitis aka “flesh-eating disease”, seborrheic dermatitis, atopic dermatitis, and graft-versus-host disease) in a subject a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to the subject with elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111, a therapeutically effective amount of a composition comprising a Nuclear Transport Checkpoint Inhibitor (NTCI) aka Nuclear Transport Modifier (NTM) (such, as for example, a NTCI including, but not limited to compositions wherein the NTCI comprising one or more NTCIs including, but not limited to SN50 having the sequence set forth in SEQ ID NO: 1 or cSN50.1 having the sequence set forth in SEQ ID NO: 2, cSN50.1 beta having the sequence set forth in SEQ ID NO: 16, or any of the NTMs disclosed herein having the amino acid sequence set forth in SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41). In one aspect the NTM can be cSN50.1 beta comprising the amino acid sequence AAVALLPAVLLALLAPCVQRDEQKLMPC (SEQ ID NO: 16). cSN50.1 beta is a cyclized peptide having the sequence of cSN50.1 with the exception that the lysine at the position 21 has been replaced by aspartic acid and the arginine residue at the position of 22 has been replaced by glutamic acid.

Accordingly, described herein is a composition for treating an inflammatory skin disease or disorder (e.g., allergic, autoimmune, autoinflammatory, microbial, metabolic, neoplastic, and posttraumatic skin disease) in a subject. The composition includes a pharmaceutically acceptable carrier and at least one (e.g., one, two, three, etc.) importin beta-selective NTM including an SSHR domain and a cargo that does not bind to any importin alpha, or at least one (e.g., one, two, three, etc.) importin alpha-selective NTM, in an amount effective for modifying entry of at least one (e.g., one, two, three, etc.) transcription factor (e.g., NFκB, AP-1, NFAT, STAT1, SREBP1a, SREBP1c, and SREBP2, and ChREBPα and ChREBPs) into a cell's (e.g., a mammalian cell's) nucleus and for treating the inflammatory disease or disorder. The at least one importin alpha-selective NTCI is a peptide or compound that binds to one or more binding pockets of an importin alpha and that modulates nuclear transport of at least one intracellular protein. Modifying entry of at least one transcription factor into a cell's nucleus includes inhibiting entry of the at least one transcription factor into the cell's nucleus. The at least one importin beta-selective NTCI can have an amino acid sequence from the group of: SEQ ID NOs: 2 and 6 (e.g., AAVALLPAVLLALLAPVQRDEQKLMP (SEQ ID NO: 40) (referred to as SM12 in Table 3). The at least one importin alpha-selective NTM can have, for example, the amino acid sequence AAVALLPAVXLAXXAPCVQRKRQKLMPC (SEQ ID NO: 41). The composition can be administered with a corticosteroid or a non-steroidal anti-inflammatory agent. In another embodiment, the composition can further include a corticosteroid or a non-steroidal anti-inflammatory agent. The non-steroidal anti-inflammatory agent can be, for example, acetaminophen or ibuprofen or calcineurin inhibitor.

Also described herein is a method of treating or preventing inflammation in a mammalian subject (e.g., a human subject having a skin disease mediated by allergic, autoimmune, metabolic, microbial, posttraumatic or neoplastic inflammation). The method includes administering a composition including a pharmaceutically acceptable carrier and at least one importin beta-selective NTM including an SSHR domain and a cargo to the mammalian subject in an amount effective for modifying entry of at least one transcription factor (e.g., NFκB, AP-1, NFAT, STAT1, SREBP1a, SREBP1c, and SREBP2, and ChREBPα and ChREBPβ) into a cell's nucleus and for treating or preventing inflammation in the mammalian subject. In the method, the at least one importin beta-selective NTM binds to and inhibits the activity of at least one importin beta. Modifying entry of at least one transcription factor into a cell's nucleus includes inhibiting entry of the at least one transcription factor into the cell's nucleus. Administration of the composition generally results in inhibition of at least one signaling pathway associated with the inflammation. The at least one importin beta-selective NTM can have an amino acid sequence from the NTM sequences disclosed herein. The composition can be administered by any suitable route, e.g., topically, orally, intravenously, or subcutaneously.

Yet further described herein is a method of treating or preventing skin disease mediated by inflammation in a mammalian subject. The method includes administering a composition including a pharmaceutically acceptable carrier and at least one agent that inhibits an interaction between at least one importin alpha (e.g., importin alpha 1, importin alpha 3, importin alpha 4, importin alpha 5 and importin alpha 7), and at least one importin beta and that modulates nuclear transport of at least one intracellular protein, to the mammalian subject in an amount effective for modifying entry of at least one transcription factor into a cell's nucleus and for treating or preventing inflammation in the mammalian subject. Typically, the at least one agent binds specifically to the at least one importin alpha and is an importin alpha-selective inhibitor.

### 2. Methods of Treating Skin Disorders

In one aspect, disclosed herein are methods of treating/inhibiting/reducing an inflammatory skin disorder (such as, for example, a skin disorder caused by microbial agents that induce microbial inflammation, allergen (i.e., allergic process), autoimmune disorder, alloimmune disorder, autoinflammatory disorder, metabolic process, neoplastic disorder, and/or physical factors and/or insults that are mediated by inflammation, including, but not limited to contact dermatitis, psoriasis, systemic lupus erythematosus, bullous dermatitis, necrotizing fasciitis aka “flesh-eating disease”, seborrheic dermatitis, atopic dermatitis, and graft-versus-host disease) or inflammatory response caused by a skin insults in a subject a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to the subject with elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111, a therapeutically effective amount of a composition comprising a Nuclear Transport Modifier (NTM) (such, as for example, a nuclear transport checkpoint inhibitor (NTCI) including, but not limited to compositions wherein the NTM comprising one or more NTMs including, but not limited to SN50 having the sequence set forth in SEQ ID NO: 1 or cSN50.1 having the sequence set forth in SEQ ID NO: 2, cSN50.1 beta having the sequence set forth in SEQ ID NO: 16, or any of the NTMs disclosed herein having the amino acid sequence set forth in SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41).

As noted herein, inflammatory skin disorders can be caused by any number on insults including, but not limited to a skin disorder caused by a microbial infection (i.e, microbial disease). It is understood and herein contemplated that inflammation is a mechanism of disease caused by infection (“microbial insult”). An inflammatory skin disorder caused by a microbial insult evolves from innate immune response to an infection due to a microbe such as, for example, a virus, bacterium, fungus, or parasite. Thus, the microbial injury caused by microbial virulence factors is aggravated by the host-produced inflammatory mediators that impede the clearance of invading microbes and add insult to organ's injury. It is understood and herein contemplated that the inflammation and its end stage, necrosis of the skin and its underlying structures can result from any microbial insult elicited by known (or unknown) virulence factors and microbial antigens. Accordingly, in one aspect, disclosed herein are methods of treating an inflammatory skin disorder; wherein the inflammatory skin disorder is caused by a microbial disease such as, for example, a virus, bacterium, fungus, and/or parasite. Adjuvant anti-inflammatory therapy is urgently needed to counteract pathogen- and host-activated proteases responsible for the skin and subcutaneous tissue necrosis due to out-of-control microbial inflammation. Such adjuvant therapy is based on anti-inflammatory and cytoprotective action of NTMs. These cell-penetrating peptides suppress host-produced mediators of inflammation responsible for massive apoptosis and hemorrhagic necrosis of the liver and dramatically improve the clearance of invading bacteria in the lungs and other organs. Accordingly, disclosed herein are methods of treating, inhibiting, reducing, and/or preventing skin disease mediated by microbial inflammation in a subject a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to the subject with elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111, a therapeutically effective amount of a composition comprising a Nuclear Transport Modifier (NTM) (such, as for example, a nuclear transport checkpoint inhibitor (NTCI) including, but not limited to compositions wherein the NTM comprising one or more NTMs including, but not limited to SN50 having the sequence set forth in SEQ ID NO: 1 or cSN50.1 having the sequence set forth in SEQ ID NO: 2, cSN50.1 beta having the sequence set forth in SEQ ID NO: 16, or any of the NTMs disclosed herein having the amino acid sequence set forth in SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41).

In one aspect, disclosed herein are methods of treating an inflammatory skin disorder; wherein the inflammatory skin disorder is caused by a viral infection, such as, for example, an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed herein are methods of treating an inflammatory skin disorder; wherein the inflammatory skin disorder is caused by a bacterial infection, wherein the bacterial infection is an infection with a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Vibrio vulnificus, Capnocytophaga canimorsus, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In some instances, the bacteria causing the bacterial infection is not Bacillus anthracis. In one aspect the bacterial infection causes Subacute bacterial endocarditis (SBE).

In one aspect, disclosed herein are methods of treating an inflammatory skin disorder; wherein the inflammatory skin disorder is caused by a fungal infection, wherein the fungal infection is an infection with a fungi selected from the group consisting of Candida albicans, Malassezia yeasts, Cryptococcus neoformans, Histoplasma capsulatum, Aspergillus fumigatus, Coccidiodes immitis, Paracoccidioides brasiliensis, Blastomyces dermitidis, Pneumocystis jirovecii, Penicillium marneffi, and Alternaria alternata.

Also disclosed herein are methods of treating an inflammatory skin disorder; wherein the inflammatory skin disorder is caused by a parasitic infection, wherein the parasitic infection is an infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Strongyloides stercoralis, Trichobilharzia regenti, Trichinella spiralis, Trichinella britovi, Trichinella nelsoni, Trichinella nativa, and Entamoeba histolytica.

It is understood and herein contemplated that while addressing the inflammatory skin disorder may alleviate symptoms of inflammatory disorder or alleviate the skin disorder caused by the microbial infection, the methods and NTMs disclosed herein will not remove the causative microbe (although such clearance could be driven by a properly regulated host immune response). It is understood and herein contemplated that any method of treating an inflammatory skin disorder comprising administering a composition comprising any of the NTCI disclosed herein can further comprise the administration of an anti-microbial agent. Examples of anti-microbial agents include any antibiotics, antibodies, small molecules, and functional nucleic acids (siRNA, RNAi, anti-sense oligonucleotides), that directly attack the infecting microbe or alter host conditions rendering the host system inhospitable to the microbe. Such agents include, but are not limited to Abacavir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Lamivudine, Lopinavir, Loviride, Maraviroc, Moroxydine, Methisazone, Nelfinavir, Nevirapine, Nexavir, Nitazoxanide, Norvir, Oseltamivir, Peginterferon alfa-2a, Penciclovir, Peramivir, Pleconaril, Podophyllotoxin, Raltegravir, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine, Clofazimine; Dapsone; Capreomycin; Cycloserine; Ethambutol (Bs); Ethionamide; Isoniazid; Pyrazinamide; Rifampicin; Rifabutin; Rifapentine; Streptomycin; Arsphenamine; Chloramphenicol (Bs); Fosfomycin; Fusidic acid; Metronidazole; Mupirocin; Platensimycin; Quinupristin/Dalfopristin; Thiamphenicol; Tigecycline (Bs); Tinidazole; Trimethoprim (Bs); aminoglycosides such as, for example, Amikacin, Gentamicin, Kanamycin, Meropenem, Neomycin, Netilmicin, Tobramycin, Paromomycin, Streptomycin, Spectinomycin, Nitazoxanide, Melarsoprol Eflornithine, Metronidazole, Tinidazole, Miltefosine, Mebendazole, Pyrantel pamoate, Thiabendazole, Diethylcarbamazine, Ivermectin, Niclosamide, Praziquantel, Albendazole, Praziquantel, Rifampin, Amphotericin B, Fumagillin, Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Efinaconazole, Epoxiconazole, Fluconazole, Isavuconazole, Itraconazole, Omadacycline, Posaconazole, Propiconazole, Ravuconazole, Terconazole, Voriconazole, Abafungin, Anidulafungin, Caspofungin, Micafungin, Aurones, Benzoic acid, Ciclopirox, Flucytosine, Griseofulvin, Haloprogin, Tolnaftate, Undecylenic acid, Crystal violet, Balsam of Peru, Orotomide, Miltefosine; ansamycins, such as, for example, geldanamycin, rifaximin, herbimycin; Carbapenems, such as, for example, Ertapenem, Doripenem, Imipenem/Cilastatin, and Meropenem; Cephalosporins, such as, for example, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, and Ceftobiprole; Glycopeptides, such as, for example Teicoplanin, Vancomycin, Telavancin, Dalbavancin, and Oritavancin; Lincosamides (Bs), such as, for example, Clindamycin and Lincomycin; Lipopeptides, such as, for example, Daptomycin; Macrolides (Bs), such as, for example, Azithromycin, Clarithromycin, Erythromycin, Roxithromycin, Telithromycin, and Spiramycin; Monobactams, such as, for example, Aztreonam; Nitrofurans, such as, for example, Furazolidone and Nitrofurantoin (Bs); Oxazolidinones (Bs), such as, for example, Linezolid, Posizolid, Radezolid, and Torezolid; Penicillins, such as, for example, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; Polypeptides, such as, for example, Bacitracin, Colistin, and Polymyxin B; Quinolones/Fluoroquinolones, such as, for example, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; Sulfonamides (Bs), such as, for example, Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), and Sulfonamidochrysoidine (archaic); Tetracyclines (Bs), such as, for example, Demeclocycline, Doxycycline, Metacycline, Minocycline, Omadacycline, Oxytetracycline, and Tetracycline; monoclonal antibodies such as, for example, Actoxumab, Atidortoxumab, Berlimatoxumab, Bezlotoxumab, Cosfroviximab, Edobacomab, Felvizumab, Firivumab, Foravirumab, Larcaviximab, Motavizumab, Navivumab, Panobacumab, Palivizumab, Porgaviximab, CR6261, Rafivirumab, Pagibaximab, Obiltoxaximab, Ibalizumab, Regavirumab, Rmab, Sevirumab, Rivabazumab pegol, Tefibazumab, Suvratoxumab, and Tuvirumab; and checkpoint inhibitors; Pembrolizumab, Nivolumab, Atezolizumab, Avelumab, Durvalumab, pidilizumab, AMP-224, AMP-514, PDR001, cemiplimab, and Ipilimumab.

In one aspect, it is understood and herein contemplated that the inflammatory skin disorder treated using the methods and NTCIs disclosed herein can be caused by an autoimmune disease. Autoimmune diseases are set of diseases, disorders, or conditions resulting from an adaptive immune response (autoreactive T cell and/or B cell response) against the host organism. In such conditions, either by way of mutation or other underlying cause, the host T cells and/or B cells and/or antibodies are no longer able to distinguish host cells, their constituents, and extracellular proteins from non-self-antigens and attack host cells (or their products) bearing an antigen for which they are specific. For example, autoreactive T lymphocytes that attack skin cells in psoriasis and the joint lining in psoriatic arthritis manifested by enthesitis and dactylitis. Autoreactive B lymphocytes that produce anti-DNA antibodies are associated with skin lesions and other organs dysfunction (eg cardiovascular system and kidneys) in lupus erythematosus. Autoreactive B and T cells usually persist due to their resistance to activation-induced cell death. Fortunately, they can be reduced or eliminated by treatment with NTM peptides in experimental model of autoimmune disease. Examples of autoimmune diseases that can cause an inflammatory skin disorder include, but are not limited to Contact Dermatitis, Graft-Versus-Host Disease, Pemphigus, Psoriasis, Rosacea, Scleroderma, Systemic Lupus Erythematosus, Achalasia, Acute disseminated encephalomyelitis, Acute motor axonal neuropathy, Addison's disease, Adiposis dolorosa, Adult Still's disease, Agammaglobulinemia, Alopecia areata, Amyloidosis, Ankylosing spondylitis, Antiphospholipid syndrome, Autoimmune angioedema, Autoimmune dysautonomia, Autoimmune urticaria, Behcet's disease, Bullous pemphigoid, Eosinophilic Granulomatosis (EGPA), Cicatricial pemphigoid, Cogan's syndrome, Cold agglutinin disease, Dermatitis herpetiformis, Dermatomyositis, Eosinophilic fasciitis, Erythema nodosum, Essential mixed cryoglobulinemia, Fibromyalgia, Granulomatosis with Polyangiitis, Graves' disease, Henoch-Schonlein purpura (HSP), Herpes gestationis or pemphigoid gestationis (PG), Hidradenitis Suppurativa (HS) (Acne Inversa), Lichen planus, Lichen sclerosus, Lupus nephritis, Lupus vasculitis, Lyme disease chronic, Microscopic polyangiitis (MPA), Mixed connective tissue disease (MCTD), Mooren's ulcer, Neonatal Lupus, Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopeni purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)).

It is understood that not all inflammatory skin disorders resulting from attack by the host immune system involve the adaptive immune response. In some instances, the innate immune response (i.e., NK cells, macrophage, dendritic cells, and innate lymphoid cells) are constitutively activated and so produced inflammatory mediators attack the host cells. Diseases where the host innate immune response attacks host cells is referred to as an “autoinflammatory disease.” In one aspect, disclosed herein are methods of treating an inflammatory skin disorder in a subject comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to the subject a therapeutically effective amount of a composition comprising a Nuclear Transport Modifier (NTM); wherein the inflammatory skin disorder is caused by an autoinflammatory disorder. Examples of autoinflammatory disorder that can cause the inflammatory skin disorders treated by the disclosed methods include, but are not limited to Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF), Tumor Necrosis Factor (TNF)-Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1ß (IL-1ß) Receptor Antagonist (DIRA) (also known as OsteomyelitisSterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behçets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome, Acute Febrile Neutrophilic Dermatosis, X-linked familial hemophagocytic lymphohistiocytosis, and Lyn kinase-associated Autoinflammatory Disease (LAID).

In one aspect, it is understood and herein contemplated that metabolic disorders can underly the inflammation that results in an inflammatory skin disorder or inflammatory symptoms on the skin. As metabolic inflammation depends on nuclear transport of at least three classes of transcription factors SREBPs and ChREBPs, and proinflammatory SRTFs, NTM peptides that target signaling pathways mediated by these transcription factors (see FIGS. 1A and B) are highly likely to be effective in these skin diseases. Accordingly, disclosed herein are methods of treating an inflammatory skin disorder, wherein the inflammatory skin disorder is caused by a metabolic syndrome or disease. In one aspect, the systemic or localized metabolic disorder can be selected from the group consisting of seborrheic acne, Gout, Skin Aging, Xanthelasma, metabolic syndrome, diabetes mellitus, obesity, Gaucher's disease, Phenylketonuria (PKU), Maple syrup urine disease (MSUD), fatty liver, hypercholesterolemia, hypertriglyceridemia, hyperthyroidism, hypothyroidism, dyslipidemia, hypolipidemia, and galactosemia.

It is understood and herein contemplated that inflammatory skin disorders can be caused by uncontrolled proliferation of certain types of skin cells or skin-infiltrating cells (i.e., neoplastic disorders and cancers). Thus, for example, disclosed herein are methods of treating inflammatory skin disorder comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to a subject with an inflammatory skin disorder a therapeutically effective amount of a composition comprising a NTM, wherein the inflammatory skin disorder is caused by uncontrolled proliferation (such as, for example, a neoplastic disorder or cancer). In one aspect, disclosed herein are methods of treating an inflammatory skin disorder caused by a neoplastic disorder or a cancer, wherein the neoplastic disorder or cancer is selected from the group consisting of Mycosis Fungoides, Sezary Syndrome, Kaposi's Sarcoma, Adult T cell Leukemia/Lymphoma, PTEN hamartoma syndrome, Familial adenomatous polyposis, Tuberous sclerosis complex, Von Hippel-Lindau disease, ovarian teratomas, meningiomas, osteochondromas, B cell lymphoma, T cell lymphoma, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, and pancreatic cancer. In some instances, such as skin T-cell lymphoma, treatment involves extracorporeal exposure of blood to UV source with appropriate sensitizing agent. NTM peptides can be added to such a therapeutic system.

It is well established that physical injury through abrasion, puncture, laceration, contusion, blunt force trauma, ischemia, surgery (including, but not limited to surgical wounds or burns), aging, aging caused by exposure to ultraviolet (UV) light, bedsores, transplant, sunburn, chemical burn, electrical burn, high temperature burn, low temperature burn can produce an inflammatory response. Some of these responses can either result in inflammation that manifests on the skin or an inflammatory skin disorder. Accordingly, disclosed herein are methods of treating an inflammatory skin disorder comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to a subject with an inflammatory skin disorder a therapeutically effective amount of a composition comprising a NTM, wherein the inflammatory skin disorder is caused by physical injury. In one aspect, the physical injury can be selected from the group consisting of abrasion, puncture, laceration, contusion, blunt force trauma, ischemia, surgery (including, but not limited to surgical wounds or burns), aging, aging caused by exposure to UV light, bedsores, transplant, sunburn, electrical burn, chemical burn, high temperature burn, low temperature burn.

The methods disclosed herein involve treating inflammatory skin disorders or symptoms from other inflammatory insults on the skin. It is understood and herein contemplated that many treatments of inflammatory conditions will involve the treatment of a wound. Thus, in one aspect, disclosed herein are methods of treating a wound comprising contacting the wound with a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. It is further understood, that by treating a wound with a therapeutically effective amount of a composition comprising a NTM not only will the wound be treated, but the time needed for the healing process can be reduced compared to untreated wounds. Thus, disclosed herein are methods of reducing the healing time of a wound comprising contacting the wound with a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

In one aspect, it is understood and herein contemplated that one way to treat a wound is through administration of the NTM subcutaneously, intramuscularly, intravenously, topically (such as, for example, through the use of salves, creams, and/or ointments), but also by impregnating bandages, dressing, sutures, drapes, surgical adhesive, and/or staples with the NTM. Thus, in one aspect, disclosed herein are medicated adhesive bandages, wound dressings, surgical drapes, sutures, salves, creams, or wound adhesives comprising a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. It is understood and herein contemplated that the medicated adhesive bandages, wound dressings, surgical drapes, staples, sutures, salves, creams, or wound adhesives disclosed herein can be used in conjunction with any of the disclosed methods of treatment. Thus, in one aspect, disclosed herein are methods of treating/inhibiting/reducing an inflammatory skin disorder (including, but not limited to inflammatory skin disorders caused by microbial disease, autoimmune disease, autoinflammatory disorder, metabolic disorder, neoplastic disorder, or physical insults that are mediated by inflammation), treating a wound, and/or reducing the healing time of a wound comprising administering to a subject with a skin disorder and/or wound the medicated adhesive bandages, wound dressings, surgical drapes, staples, sutures, salves, creams, or wound adhesives disclosed herein.

3. Methods of Treating Physical Factors and/or Physical Insults

Many inflammatory conditions result from physical injuries mediated by inflammation (such as, for example abrasion, puncture, laceration, contusion, blunt force trauma, ischemia, hemorrhagic stroke, surgery (including, but not limited to surgical wounds or burns), transplant, bedsores, electric burn, sunburn, chemical burn, high temperature burn, low temperature burn, radiation injury, and skin aging). As noted above, the NTMs disclosed herein can target the nuclear transport shuttles, Imp α5 and Imp β1, that translocate SRTFs and SREBPs to the nucleus and control signal transduction pathways, which culminate in genomic reprogramming. Thus, the novel forms of immunotherapy disclosed herein that targets nuclear import as described herein can arrest inflammation-driven destruction associated with these physical injuries. Accordingly, in one aspect, disclosed herein are methods of treating inflammation caused by physical injury (such as, for example, abrasion, puncture, laceration, contusion, blunt force trauma, ischemia, hemorrhagic stroke, surgery (including, but not limited to surgical wounds or burns), transplant, sunburn, chemical burn, high temperature burn, low temperature burn) comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to the subject with elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111, a therapeutically effective amount of a composition comprising a Nuclear Transport Modifier (NTM) (such, as for example, a nuclear transport checkpoint inhibitor (NTCI) including, but not limited to compositions wherein the NTM comprising one or more NTMs including, but not limited to SN50 having the sequence set forth in SEQ ID NO: 1 or cSN50.1 having the sequence set forth in SEQ ID NO: 2, cSN50.1 beta having the sequence set forth in SEQ ID NO: 16, or any of the NTMs disclosed herein having the amino acid sequence set forth in SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41).

It is understood and herein contemplated that many inflammatory conditions resulting from inflammatory injury or physical injuries mediated by inflammation (such as, for example abrasion, puncture, laceration, contusion, blunt force trauma, ischemia, hemorrhagic stroke, surgery (including, but not limited to surgical wounds or burns), transplant, bedsores, electric burn, sunburn, chemical burn, high temperature burn, low temperature burn, radiation injury, and skin aging), said treatments will involve the treatment of a wound. Thus, in one aspect, disclosed herein are methods of treating a wound comprising contacting the wound with a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. It is further understood, that by treating a wound with a therapeutically effective amount of a composition comprising a NTM not only will the wound be treated, but the time needed for the healing process can be reduced compared to untreated wounds. Thus, disclosed herein are methods of reducing the healing time of a wound comprising contacting the wound with a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. In some aspect, the NTM can be administered orally, topically, intravenously, and/or a medicated adhesive bandage, wound dressing, surgical drape, suture, salve, cream, or wound adhesive comprising a therapeutically effective amount of a composition comprising a Nuclear Transport Modifier (NTM).

In one aspect, it is understood and herein contemplated that one way to treat a wound is through administration of the NTM subcutaneously, intramuscularly, intravenously, topically (such as, for example, through the use of salves, creams, and/or ointments), but also by impregnating bandages, dressing, sutures, drapes, surgical adhesive, and/or staples with the NTM. Thus, in one aspect, disclosed herein are medicated adhesive bandages, wound dressings, surgical drapes, sutures, salves, creams, lotions, or wound adhesives comprising a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. It is understood and herein contemplated that the medicated adhesive bandages, wound dressings, surgical drapes, staples, sutures, salves, creams, or wound adhesives disclosed herein can be used in conjunction with any of the disclosed methods of treatment. Thus, in one aspect, disclosed herein are methods of treating/inhibiting/reducing a physical injury mediated by inflammation (including, but not limited to inflammatory skin disorders caused by physical insults that are mediated by inflammation), treating a wound, and/or reducing the healing time of a wound comprising administering to a subject with a skin disorder and/or wound the compositions comprising administering to the subject a therapeutically effective amount of a composition comprising a NTM and/or any medicated adhesive bandages, wound dressings, surgical drapes, staples, sutures, salves, lotions, creams, or wound adhesives disclosed herein.

### 4. Methods of Assessing the Efficacy of a Treatment Regimen or a Subject's Responsiveness to a Treatment Regimen.

In one aspect, disclosed herein are methods of assessing a treatment regimen or a subjects responsiveness to a treatment regimen for an inflammatory skin disease or inflammatory skin disorder caused by a microbial, allergic, autoimmune, constitutive, metabolic, neoplastic and/or physical insults (such as, for example, atopic dermatitis) in a subject the method comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates that the treatment regimen is not effective against the inflammatory skin disease or inflammatory skin disorder caused by a microbial, allergic, autoimmune, constitutive, metabolic, neoplastic and/or physical insults or the subject is not responsive to the treatment regimen.

Also disclosed herein are methods of assessing a treatment regimen or a subjects responsiveness to a treatment regimen, wherein the treatment regimen comprises a composition comprising a nuclear transport modifier (NTM) (such, as for example, a nuclear transport checkpoint inhibitor (NTCI) including, but not limited to compositions wherein the NTM comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

In one aspect, disclosed herein are methods of assessing a treatment regimen or a subject's responsiveness to a treatment regimen, wherein the inflammatory skin disease or inflammatory skin disorder is caused by microbial agents, autoimmune disorder, alloimmune disorder, allergens (i.e., allergic process), autoinflammatory disorder, metabolic process, neoplastic/oncogenic disorder, or physical insults that are mediated by inflammation (such as, for example, atopic dermatitis).

### 5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### 6. Homology/Identity

It is understood that one way to define any known variants and derivatives or those that might arise, of the disclosed genes, proteins, herein is through defining the variants and derivatives in terms of homology to specific known sequences. For example SEQ ID NO: 2 sets forth a particular sequence of an NTM (cSN50.1). Specifically disclosed are variants of these and other genes- and proteins-derived peptide sequences herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins, peptides or nucleic acids, such as genes encoding proteins. For example, the homology can be calculated after aligning the two sequences so that the homology is at its highest level. As used herein, sequence homology is used interchangeably with sequence identity.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 7. Peptides

### a) Peptide Variants

As discussed herein there are numerous variants of the NTCI aka NTM that are known and herein contemplated. Peptide variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 4 and 5 and are referred to as conservative substitutions.

TABLE 4
Amino Acid Abbreviations

| Amino Acid | Abbreviations | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 5
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn;Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 5, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed protein-derived peptides herein is through defining the variants and derivatives in terms of homology/identity to specific known sequences. For example, SEQ ID NO: 2 sets forth a particular sequence of cSN50.1. Specifically disclosed are variants of these and other proteins herein disclosed which have at least, 70%, 75%, 80%, 85%, 90%, 91%, 92%, 93%, 94%, 95%, 96%, 97%, 98%, 99%, 99.1, %, 99.2%, 99.3%, 99.4%, 99.5%, 99.6%, 99.7%, 99.8%, 99.9%, or 100% sequence identity to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 4 and Table 5. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site-specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. Stapled alpha-helical sequence of signal-sequence hydrophobic region can be used to stabilize its membrane-translocating conformation in NTM.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Genomic Control of Inflammation in Experimental Atopic Dermatitis

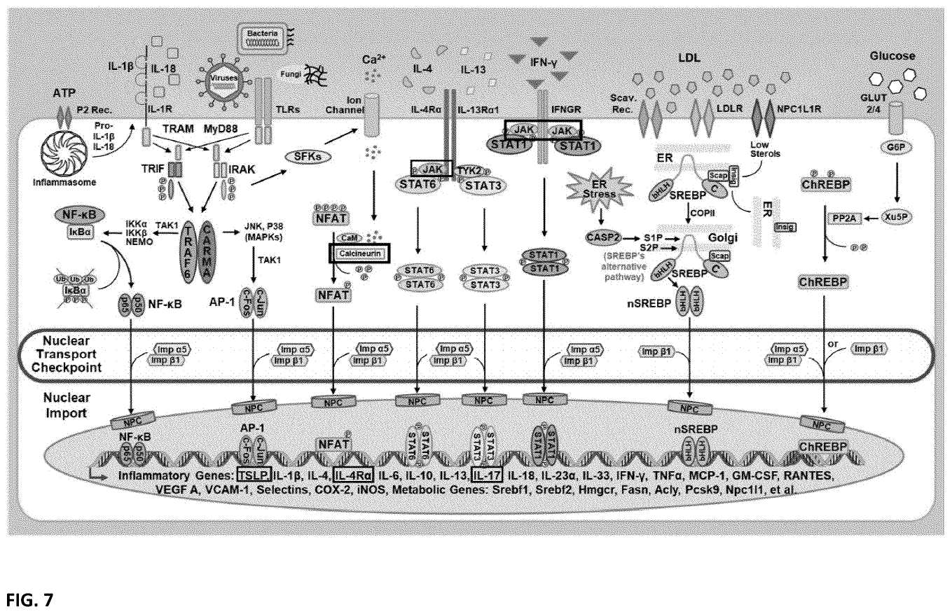

An alternative to glucocorticoids therapy is topical treatment with Nuclear Transport Checkpoint Inhibitor (NTCI). This anti-inflammatory cell-penetrating peptide targets two nuclear transport shuttles, cytoplasmic adaptor proteins termed importin α5 and importin β1. Targeting these two proteins that comprise the nuclear transport checkpoint (see FIG. 7) produces a broad-spectrum anti-inflammatory activity through silencing of at least 50 genes encoding mediators of inflammation and metabolism. Their expression depends on the nuclear transport/translocation of an entire set of proinflammatory Stress-Responsive Transcription Factors (SRTFs), which include NF-κB, cFos/cJun, STAT1, and NFATs. Most recently, we found that the nuclear import of STAT3, the transcription factor involved in allergic inflammation, is inhibited by targeting importin α5 with NTCI in primary human keratinocytes derived from adult skin (unpublished data). In parallel, Metabolic Transcription Factors (MTFs) are also translocated to the cell's nucleus. MTFs comprise Sterol Regulatory Elements-Binding Proteins (SREBPs) translocated solely by importin β1, and Carbohydrate Response Element-Binding Proteins (ChREBPs) shuttled either by importin α5 that recognizes the NLS “zip code” or importin β1 that binds to the Helix-Loop-Helix Leucine Zipper-binding site (see FIG. 7). NTCI, the cSN50.1 peptide, stopped not only SRTFs and SREBPs but also ChREBPs on their path to nucleus by targeting the two components of the nuclear transport checkpoint, importin α5 and importin β1. Hence, the inflammatory regulome in the genome comprising the multiple genes that encode the mediators of allergic, autoimmune, microbial, and metabolic inflammation, cannot be activated. We postulated that AD can be treated by NTCI through inhibition of nuclear signaling, thereby suppressing inflammatory injury to the skin.

The experimental model of AD caused by topical administration of MC903, the low calcemic analog of Vitamin D3, was introduced by Pierre Chambon's group after calcipotriol was found to cause the signs of AD in clinical trials for the treatment of skin psoriasis. MC903, applied topically to mouse ears, induces the characteristic signs of AD consisting of swelling, and T_{H2} type inflammation manifested in a dermal infiltrate. The pathognomonic features of AD were linked to epidermal keratinocytes expressing Thymic Stromal Lymphopoietin (TSLP). Its genetic ablation prevented AD-like skin changes upon challenge with MC903. Moreover, TSLP role in promoting T_{H2} response and driving the pathogenesis is critical in the mechanism of AD.

Keratinocytes are strategically positioned in the outermost layer of the skin, the epidermis. They form a multilayered structure to maintain the functional integrity of the skin, which depends on three compartments within the epidermis: (i) the air-liquid barrier, (ii) the liquid-liquid barrier, and (iii) the immune defense barrier. Langerhans cells comprise the latter barrier. Their dendritic projections abut the keratinocytes, bridged by Tight Junctions (TJs), thereby demarcating the fluid microenvironment of the skin and favoring intercellular communication during the inflammatory response. Keratinocytes, also named “cytokinocytes”, contribute to immune responses in skin.

Keratinocytes sense and respond to proinflammatory insults by activating the signaling pathways to the nucleus, the site of the inflammatory regulome (see above) 7. Therein, SRTFs and MTFs reprogram the inflammatory regulome and activate multiple genes that encode a myriad of inflammatory mediators. They encompass cytokines, chemokines, their receptors, cell adhesion molecules, as well as extracellular and intracellular signaling intermediates.

TSLP expressed by epidermal keratinocytes promotes T_{H2} response and drives the pathogenesis of AD. Therefore, we aimed to establish whether NTCI can suppress TSLP and other genes involved in the allergic skin inflammation underlying AD.

We show that NTCI effectively reduces the cardinal signs of AD in an experimental model. To our knowledge, this study is the first preclinical proof of concept for the genomic control of AD by NTCI at the nuclear transport level. It can be extended to other inflammatory skin diseases. Its translational value is being evaluated in the ongoing Phase 2 Clinical Trial for mild to moderate AD (AMTX-100 CF, NCT04313400).

### a) Results

### (1) Genomic Control of Experimental Atopic Dermatitis by a Nuclear Transport Checkpoint Inhibitor (NTCI)

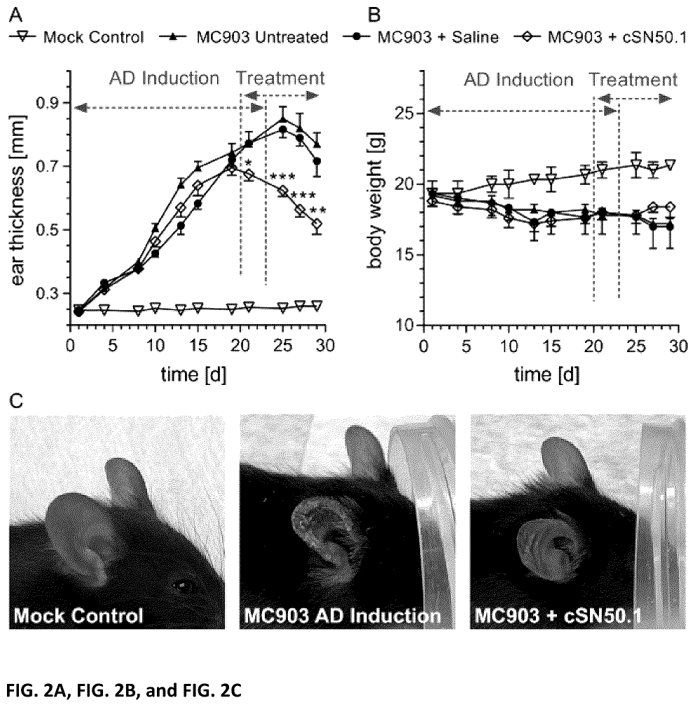

AD was induced by a topical application of MC903 to both sides of the right ear for 23 days (FIG. 1). Topical treatment with an aqueous solution of NTCI, cell-penetrating cSN50.1 peptide, 200 μg (66 nmoles, administered twice daily to the right ear) was initiated on day 20 and continued for 9 days. The right ear of MC903-challenged mice became progressively swollen until treatment with NTCI significantly reduced skin edema, one of the chief signs of inflammation (FIG. 2A). Moreover, skin lesions, prominently displayed in the ear during the MC903 induction, were healed after 9 days of treatment with NTCI (FIG. 2C).

Thus, treatment with NTCI effectively reduced existing skin inflammatory signs, such as swelling, discoloration, and scaling. A troubling side effect of AD induction with MC903 is body weight loss. Notably, the animals treated with NTCI maintained their body weight (FIG. 2B), indicating a lack of NTCI's general toxicity. This is consistent with the lack of apparent toxicity associated with long-term NTCI treatment. The lack of apparent toxicity of NTCI was also independently attested prior to the submission of the clinical trial protocol to the FDA (NCT04313400).

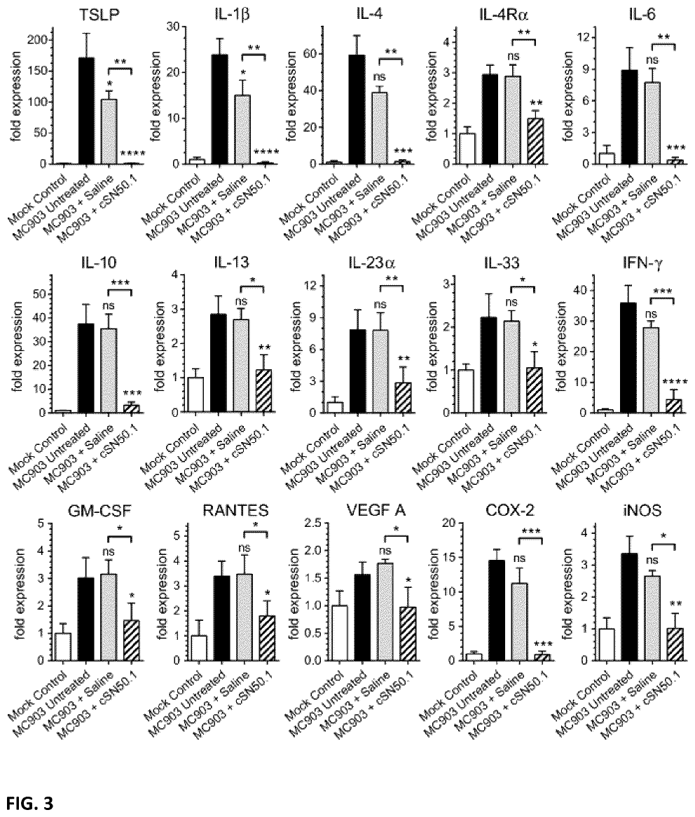

Gene expression profiling of skin obtained from animals challenged with MC903 and left untreated was compared to skin obtained from animals challenged with MC903 and treated with saline, and animals challenged with MC903 and treated with NTCI. Gene expression in skin obtained from mock control animals, which were not challenged with MC903, is also shown for comparison (FIG. 3).

The development of skin lesions in MC903-challenged ears was accompanied by the induction of genes encoding the mediators of allergic skin inflammation, including TSLP, the inducer of the T_{H2} response, and its mediators, IL-4, IL-4Rα, IL-13 along with IL-6, IL-23α, IL-33, IFN-γ, and other cytokines, chemokines, growth factors, and intracellular signaling intermediates. TSLP activates Langerhans Cells, which initiate an adaptive T_{H2} response, thereby demonstrating a central function of keratinocytes-derived TSLP in AD development. In addition, experimental AD was associated with increased expression of genes encoding the chemokine RANTES, as well as two intracellular signaling intermediates, COX-2, and iNOS, responsible for the synthesis of prostaglandins and nitric oxide (NO), respectively. The latter, together with VEGF A, are significant factors in the microvascular endothelial injury associated with skin inflammation manifested by swelling and itching.

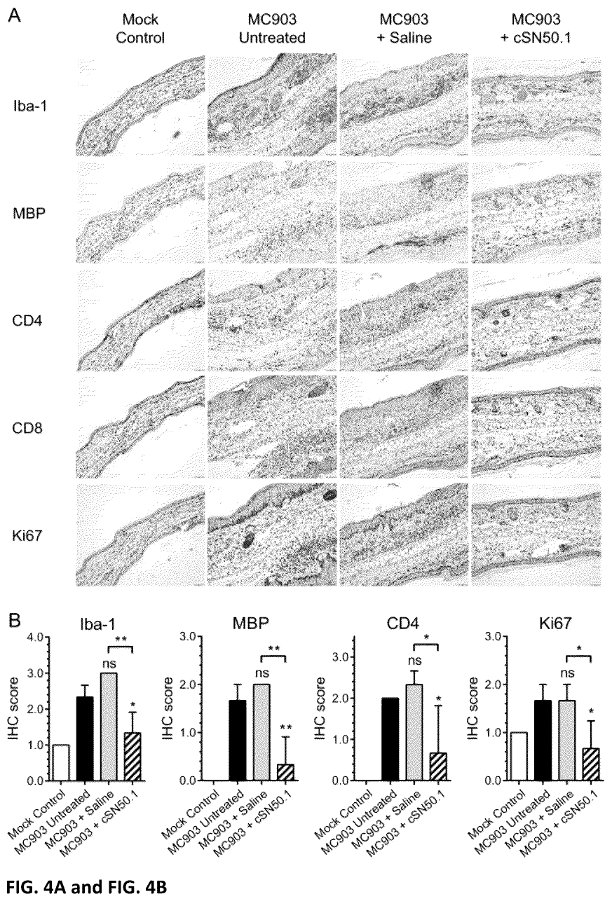

MC903-induced AD was manifested by infiltration of the skin with macrophages, eosinophils, and T lymphocytes, including CD4+ T cells. Inflammatory skin lesions consisted predominantly of macrophages and eosinophils, combined with an influx of CD4+ T cells, and significant proliferation of Ki67-positive cells in the basal cell layers of the epidermis (FIG. 4A). Immunohistochemical analysis also reflected NTCI control of the cellular effectors of AD. Thus, the major criteria for the experimental AD were controlled by NTCI. Consequently, we recorded a striking reduction of experimental AD manifested by redness, swelling, and infiltration of immune cells involved in skin inflammation.

Skin infiltration by eosinophils, macrophages, and T cells in response to MC903 was suppressed by NTCI treatment as documented by a semiquantitative analysis of immunohistochemical (IHC) staining (FIG. 4B). T cell infiltrates consisted primarily of CD4+ T cells with a paucity of CD8+ T cells. Strikingly, the proliferation of Ki67-positive cells in the basal zone of the epidermis was also attenuated. We show that the cellular immunopathology of experimental AD is tightly linked to the genomic burst of the key mediators of skin inflammation. Taken together, these cellular mediators were suppressed by NTCI treatment thereby demonstrating a profound attenuation of the skin inflammatory response.

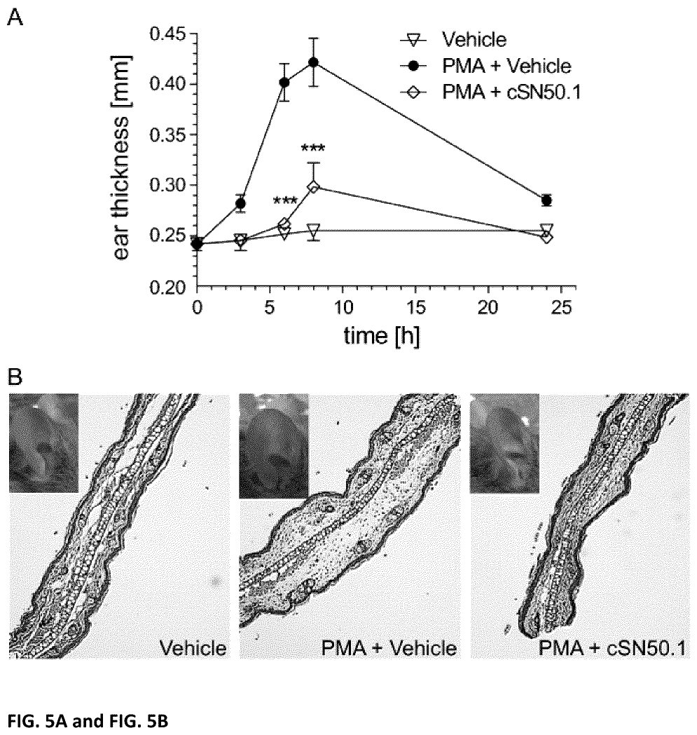

Significantly, NTCI also suppressed skin inflammation induced by another agent, Phorbol-12-Myristate-13-Acetate (PMA), which produces severe thickening of the pinna (FIG. 5). Topically-applied PMA causes chronic skin inflammation mediated by Kit, a receptor tyrosine kinase required for mast cell accumulation. The resolution of inflammation in this second model of experimental AD controlled by NTCI, is consistent with our prior discovery of its effectiveness in suppressing the PMA-induced nuclear transport of SRTFs comprising NF-κB, cFos/cJun, NFAT, and STAT1 in human T cells. Thus, NTCI suppression of cardinal manifestations of MC903-induced AD (skin swelling, eczematous lesions, and dermal infiltrate), and PMA-induced skin inflammation attests to an effective and broad-range action of NTCI in these two preclinical settings.

### b) Discussion

We wish to report the NTCI, cell-penetrating cSN50.1 peptide, as a novel agent for treatment of AD, a recurrent inflammatory skin disease afflicting a sizable population of children and adults worldwide. A major step in understanding the mechanism of AD is the outcome of NTCI topical treatment of skin lesions caused by repeated challenge with MC903 (calcipotriol) for 23 days in an experimental model of AD. This vitamin D3 analog was found to induce the signs of AD during its clinical trial in patients with psoriasis. Notably, TSLP-deficient mice did not develop AD when challenged with MC903 thereby assigning a major role to the gene encoding TSLP in the mechanism of AD.

NTCI suppresses the gene encoding TSLP in mice challenged with MC903 (see FIG. 3), thereby providing a new line of evidence for the key role of TSLP in the mechanism of AD. Moreover, success of NTCI treatment also lies in the control of genes encoding other significant mediators of the T_{H2} response in AD, such as IL-4, its receptor, IL-4Rα, and IL-13. In addition, follicular helper T cells (T_{FH}) are essential for humoral immunity and T-cell memory in atopic diseases. The ‘Atopic March” orchestrated by TSLP, IL-4, its receptor IL-4R, that also binds IL-13, extends to AD, Asthma, and Eosinophilic Esophagitis. The migration of T_{FH} cells depends on the chemokine receptor CXCR5 (CD185) regulated by SRTFs whose nuclear translocation is controlled by NTCI. Furthermore, silencing of the gene encoding IL-23α, is significant as this cytokine induces AD-like inflammation in CCR2-deficient mice. The gene encoding VEGF A, known as vascular permeability factor, was also suppressed by NTCI in the MC903-challenged skin explaining at least in part its reduction of swelling. Of note, the VEGF also contributes to the microvascular endothelial injury underling skin inflammation in psoriasis and other skin disorders. Taken together, this outcome presents NTCI as an effective and safe topical treatment of eczema and other inflammatory skin diseases.

Neuropathic itch is a significant sign of neurogenic inflammation in AD and other chronic skin inflammatory diseases. In addition to triggering T_{H2}-mediated allergic response, keratinocyte-derived TSLP activates cutaneous sensory neurons to induce itch. Striking suppression of TSLP expression in the skin challenged by MC903 (see FIG. 3) explains the itch-reducing potential of NTCI. Moreover, its inhibition of nuclear translocation of STAT3 in human keratinocytes (unpublished results), and by extension, in sensory neurons, can also contribute to the itch-reducing action of NTCI. Both STAT3 and importin α5, which is the target of NTCI, are involved in lesioned peripheral nerve, as reported by Ben-Yaakov and colleagues.

The suppression of two other genes expressed in inflamed skin, IL-1β, and IL-6 (see FIG. 3) is also significant. These cytokines are responsible for the generalized inflammatory response known as the “acute phase protein response” associated with Endoplasmic Reticulum (ER) stress. Thus, NTCI counteracts both the localized and, systemic inflammatory response in AD. Moreover, the uncontrolled action of IL-1β is linked to an inflammatory disorder of the skin and bones caused by the loss-of-function mutation of IL-1 receptor antagonist. IL-Iβ also mediates ILC2-independent “spontaneous AD” in mice with defective skin barrier.

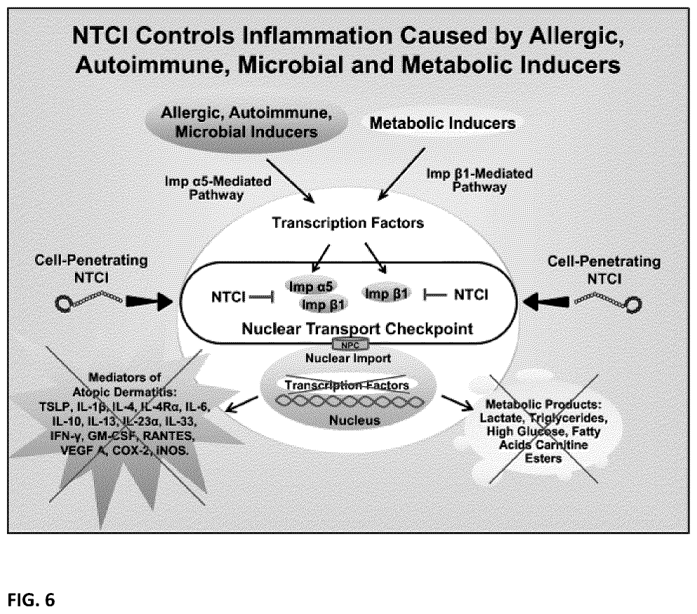

The discovery of the nuclear transport checkpoint inhibitor's role in the control of the key genes underlying the mechanism of AD is of particular significance. We submit that AD is chiefly mediated by the proinflammatory SRTFs (FIG. 6). These transcription factors encompass NF-κB. AP1 (cFos and cJun), STAT1, and NFATs as well as STAT3, the transcription factor involved in AD and other diseases mediated by allergic inflammation.

In contrast to SRTFs and MTFs (MW>45 kDa), the smaller transcription factors (MW<45 kDa), some of which are essential to cell survival and maintenance, have “a free pass” to the nucleus, providing homeostatic control of the cell's function and lifespan. To this end, the smaller transcription factors cross the nuclear envelope while bypassing the nuclear transport checkpoint targeted by NTCI. Accordingly, we found that NTCI treatment did not alter the expression of the five housekeeping genes (Gusb, Hprt1, Hsp90ab1, Gapdh, and Actb).

Our findings of significant suppression of the genes encoding TSLP and IL-1β have therapeutic implications for other human skin diseases caused by an inborn overproduction of these cytokines. The uncontrolled TSLP signaling plays a key role in patients with Netherton Syndrome, a rare hereditary skin disorder manifested by a susceptibility to AD, scaling skin, and hair abnormalities. The uncontrolled action of IL-1β is linked to an inflammatory disorder of the skin and bones caused by the loss-of-function mutation of IL-1 receptor antagonist. These and other autoinflammatory diseases of the skin and other organs are mediated by constitutive inflammation 7. The afflicted patients can benefit from NTCI-based therapy suppressing TSLP, IL-1β, and other relevant and excessively acting mediators.

Clearly, the NTCI reported in this study represents a new class of broad-spectrum anti-inflammatory agents for topical (localized) and, if needed, systemic therapy. We are not aware of any published study of AD, which applied a similar method of controlling gene transcription by the inhibition of nuclear transport to suppress the expression of the key genes involved in the development of AD through the T_{H2} response. Among the other known broad-spectrum anti-inflammatory therapies, the glucocorticoids suppress inflammatory regulome via the action of their cognate nuclear receptor, which functions as transcription factor. However, glucocorticoids cause hyperglycemia, hyperlipidemia, osteoporosis, skin atrophy, and immunosuppression. In striking contrast, NTCI reduces blood glucose, cholesterol, and triglycerides, and increases the innate immunity-mediated clearance of bacteria.

These points are relevant for the link between AD and microbial inflammation or metabolic inflammation. First, AD predisposes skin to infections with Staphylococcus aureus. The staphylococcal immunotoxin, Staphylococcal Enterotoxin B (SEB), also known as superantigen, stimulates IL-13 production, which is viewed as an etiologic factor in infants with AD. NTCI suppressed the expression of IL-13 in the skin in our study (see FIG. 3). NTCI can suppress staphylococcal immunotoxin-associated flare ups of AD consistent with our studies demonstrating NTCI control of SEB-induced toxic shock and acute respiratory distress syndrome. Second, AD is associated with Metabolic Syndrome in pediatric and adult patients. Its spectrum encompasses obesity, hyperglycemia, hyperlipidemia, fatty liver, atherosclerosis, and hypertension. In the preclinical model of Metabolic Syndrome, due to the genetic ablation of the LDL receptor (ldlr^{-/-}), combined with the Western Diet, NTCI controlled the metabolic hypersensitivity to a sublethal dose of LPS, a well-known inducer of microbial inflammation. Thus, NTCI can also control AD, which is associated with inflammation caused by autoimmune, microbial, and metabolic inducers (see FIG. 6).

In summary, we successfully enabled NTCI to control the proinflammatory transcriptional mechanism of AD, the most common skin disease in the world. Our findings are of significant relevance to millions of individuals worldwide displaying signs of AD as well as other skin disorders that depend on the nuclear transport of transcription factors responding to inflammatory stress in human keratinocytes and immune cells. Our results provide a mechanistic blueprint for the translational application of NTCI (AMTX-100 CF), which is undergoing Phase 1/2 clinical trial for mild to moderate AD (NCT04313400).

### c) Materials and Methods

(1) The Synthesis and Purification of Cell-Penetrating Nuclear Transport Checkpoint Inhibitor, cSN50.1 Peptide

Our cell-penetrating NTCI peptide, cSN50.1 (AAVALLPAVLLALLAPCVQRKRQKLMPC, SEQ ID NO: 2) was synthesized as described elsewhere. Briefly, the peptide chain was assembled through Solid Phase Peptide Synthesis (SPPS) according to standard Fmoc chemistry protocols using an automated peptide synthesizer FOCUS XC (AAPPTec, Louisville, KY). Crude peptides were removed from the resin with a TFA cleavage cocktail and purified by dialysis against double-distilled water in 1 KDa membrane (Spectra/Por 7; Spectrum Laboratories, Rancho Dominguez, CA). The purity and structure of the final products were verified respectively by an analytical C18 RP HPLC (Beckman Coulter GOLD System, Brea, CA) and MALDI mass spectroscopy (Voyager Elite; PerSeptive Biosystems, Framingham, MA).

### (2) Animal Studies

Animal experiments were carried out in compliance with the ARRIVE guidelines and in strict accordance with the Guide for the Care and Use of Laboratory Animals of the US National Institutes of Health, and submitted protocols were approved by the Vanderbilt University Institutional Animal Care and Use Ethics Committee (Permit Number: A3227-01). Mice were closely monitored during the course of experiments and euthanized by Isoflurane inhalation followed by cervical dislocation at the experimental endpoint.

### (3) The Experimental Model of Atopic Dermatitis (Eczema)

Atopic dermatitis (AD)-like phenotype was induced in 8-week-old C57BL/6 female mice (The Jackson Laboratory) by topical application of the Vitamin D3 analog, calcipotriol (MC903). The experimental groups (Mock Control, n=3; MC903 Untreated, n=5; MC903+Saline, n=5; and MC903+cSN50.1, n=5) were selected using a double blinded randomization method.

We used saline as a control representing a vehicle for all the NTCI peptide used in this study. This vehicle control is used in FDA-approved clinical studies of human subjects in which the tested new drug is administered, including therapeutic peptides. Each experiment was performed twice to assure statistical significance and experimental reproducibility.

The Treatment Protocol (see FIG. 1 for a graphic representation of the model and treatment protocol): the initiation of AD-like phenotype induction began with a bolus dose of 6 nmoles MC903 (Millipore-Sigma #C4369) applied topically to the right ear (3 nmoles on each side). Repeated exposure to the irritant (once-a-day, Q.D.) was initiated on day 3 with 2 nmoles of MC903 (1 nmole on each side) and the challenge was continued for 21 days. NTCI treatment was introduced on day 20 as a twice daily (B.I.D.) topical application of its saline solution (10 μl of 10 mg/ml on each side) and was continued for 9 days. The control group of mice received a topical saline treatment (10 μl) on both sides of the right ear. On day 29, all mice were euthanized by over inhalation of isoflurane followed by cervical dislocation. 3-4 samples of the right ear (3 mm in diameter) were collected postmortem from auricula using a sterile disposable biopsy punch. One sample was fixed in 10% neutral-buffered formalin for histopathology and immunohistochemistry studies, remaining samples were snap frozen and stored at −80° C. for total RNA extraction.

### (4) PMA-Induced Skin Inflammation

Skin inflammation was induced in 8-week-old C57BL/6 female mice (The Jackson Laboratory) by topical application of the Phorbol-12-Myristate-13-Acetate (PMA, Millipore-Sigma #524,400). The experimental groups (Vehicle, n=5; PMA, n=5; and PMA+cSN50.1, n=5) were selected using a double blinded randomization method as described before. A single dose of PMA (2 nmoles in 10 μl EtOH) was administered topically on each side to the right ear. Topical treatment with NTCI, cSN50.1 peptide (16.7 nmoles in 10 μl EtOH on each side of the right ear) or vehicle (10 μl EtOH on each side) was administered 30 min before and 3, 6, and 8 h. after PMA challenge. The unchallenged control group of mice was treated topically with vehicle only (10 μl EtOH) following the same treatment schedule. Ear thickness was measured at 30 min before (“0 h” timepoint) and 3, 6, 8 and 24 h. after PMA challenge. 3 mm ear punch biopsies were collected at 8 h. after PMA challenge from treated area under mild anesthesia (5% isoflurane). Ear samples were fixed in 10% neutral-buffered formalin for histopathology and immunohistochemistry studies. 24 h. after PMA challenge, all mice were, euthanized by over inhalation of isoflurane followed by cervical dislocation.

### (5) Gene Expression Assay by Real-Time Quantitative Reverse Transcription PCR

The mouse ear punch biopsies were disrupted in lysis buffer on ice using a Dounce hand homogenizer. Total RNA was isolated using NucleoSpin RNA Plus kit (Macherey-Nagel, Germany) according to the manufacturer's instructions. RNA concentration and purity were determined using a NanoDrop One spectrophotometer (Thermo Fisher Scientific). 1 μg of the obtained RNA was reverse-transcribed using an iScript cDNA synthesis kit (Bio-Rad, CA). A real-time quantitative reverse transcription PCR was carried out in a 96-well plate on a QuantStudio 3 instrument using the Taqman Fast Advanced Master Mix (Thermo Fisher Scientific) according to the manufacturer's protocol. The FAM-labeled probes for mouse genes were obtained from Thermo Fisher Scientific. The raw Ct values were converted into relative expression levels using Livak's methods (2^{ΔΔCt}) with the 18S gene as a reference, and the Mock Control group as a calibrator (control). Converted Ct values were used for statistical analysis.

### (6) Histology and Immunohistochemistry

Pinna samples were collected by the postmortem punch biopsy of the skin (see above) and fixed overnight in 10% neutral buffered formalin, routinely processed, embedded in paraffin, sectioned at 5 μm and stained with Hematoxylin and Eosin (H&E). Immunohistochemistry analyses with antibodies against macrophages (Iba-1; FujiFilm, 019-19,741), eosinophils (MBP; Mayo Clinic, MR-14.7), CD4 (SYSY, HS-360 017), CD8 (SYSY HS-361 003), and Ki67 (Abcam, ab 16667) were performed on the Leica Bond Max (Leica Biosystems Inc. Buffalo Grove, IL) following standard protocols in the Translational Pathology Shared Resource at Vanderbilt University Medical Center. Briefly, all assays were antigen retrieved using an Epitope Retrieval 2 solution for 20 min except MBP (Epitope Retrieval 1 solution for 15 min). Leica polymer was used as a secondary antibody for Iba-1, CD8, and Ki67 while a rat secondary antibody (Vector BA-4001) was used for MBP and CD4. The Bond Polymer Refine Detection system was used for visualization. Immunohistochemical stains were evaluated by a veterinary pathologist (K.N.G-C and K.L.B) blinded to the composition of the groups.

### (7) Statistical Analysis

Normal distribution of data sets was verified using normal probability plot (q-q) and Kolmogorov-Smirnov Normality Test. A statistical analysis was performed using tools built-in Prism 6 software (GraphPad). Gene expression profile in ear skin samples of C57Bl/6 mice as well as semiquantitative IHC scores were analyzed by ordinary one-way ANOVA with an uncorrected Fisher's LSD test for a multiple comparison. The data is presented as a mean±SEM. p values of <0.05 were considered significant.

### 2. Example 2: Cytoprotection of Human Keratinocytes Through Silencing Proinflammatory Genes by the Cell-Penetrating Nuclear Transport Checkpoint Inhibitor

### a) Results and Discussion

Using the new generation of the NTCI, cell-penetrating cSN50.1 peptide, we established the genomic control of the experimental AD induced by a repeated skin challenge with calcipotriol to the earlobe of the C57/BL/6 mice for 23 days. The NTCI in an aqueous solution applied to the skin for 8 days reduced skin swelling, the infiltration of macrophages, eosinophils, and CD4+ lymphocytes, and suppressed proliferating Ki67-positive cells in the basal zone of the epidermis. Strikingly, the NTCI also suppressed 15 genes encoding the mediators of allergic skin inflammation.

Here, we explored the transcriptional mechanism of skin inflammation in the cell-based system, a culture of primary human adult skin-derived keratinocytes (KCs) stimulated with the inflammatory cytokine duo of TNF-α and IL-17 or with Phorbol-12-Myristate-13-Acetate (PMA), a known skin irritant and carcinogen. The KCs, also termed “cytokinocytes”, detect and respond to the proinflammatory insults through the activation of the transcriptional signaling pathways to the cell's nucleus. Therein, the inflammatory regulome is activated. It comprises the promoters and enhancers of the genes encoding the mediators of inflammation. Among these genes, TSLP is responsible for the development of calcipotriol-induced AD by promoting the Th2 response. Generally, TSLP mediates type 2 immunity at barrier surfaces and has been linked to the widespread allergic and inflammatory diseases of the skin, airways, and gut.

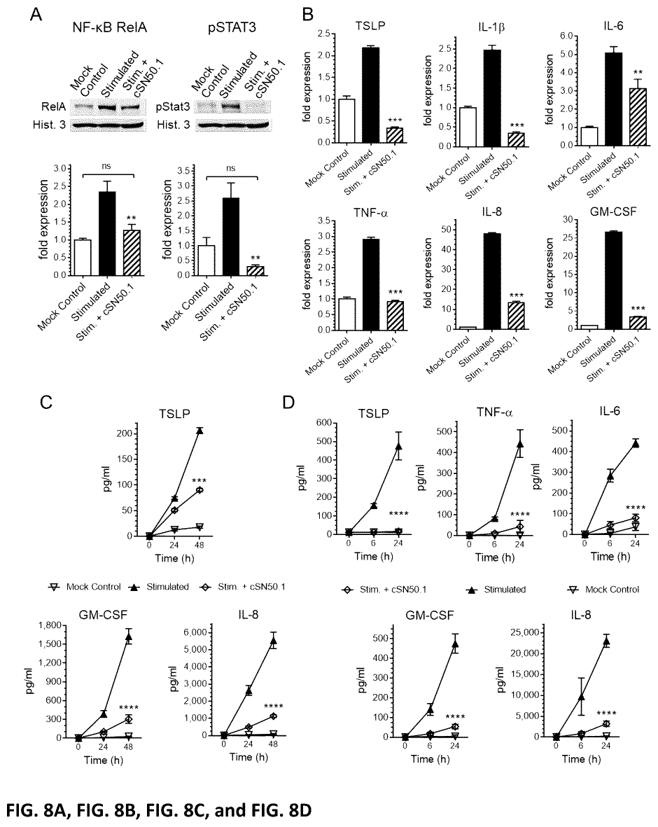

As the expression of TSLP-encoding gene in allergic diseases is regulated by the NF-κB and STAT3, we asked whether the nuclear translocation of these two acute phase response transcription factors (TFs) is inhibited by the NTCI in human KCs. Indeed, we found that the NTCI inhibited the nuclear transport of these two TFs in stimulated cells as compared to the untreated human KCs (FIG. 8A). This NTCI-imposed selective nuclear transport blockade caused the striking suppression of the key genes mediating AD and other inflammatory skin diseases. They include the gene encoding TSLP, as well as the three acute phase inflammatory mediators, IL-1β, IL-6, TNFα, and the genes encoding GM-CSF (CSF2), and IL-8 (CXCL8). The latter two contribute to the mobilization and trafficking of eosinophils, monocytes/macrophages, and other myelomonocytic cells in the dermal infiltrate (FIG. 1B).

In agreement with these genomic results, proteomic analysis of the culture medium of stimulated human KCs showed that the NTCI also suppressed the time-dependent production and release of TSLP, GM-CSF, and IL-8 proteins (FIG. 8C). Their concentration was significantly reduced reflecting the cognate gene suppression of these inflammatory mediators (FIGS. 8A and 8B). Thus, the concordance of the gene expression and protein production in human KCs (FIGS. 8A and 8B) is especially noteworthy in the case of TSLP that promotes the Th2 response.

We expanded this study to another agonist, Phorbol-12-Myristate-13-Acetate (PMA), which causes chronic skin inflammation mediated by Kit, a receptor tyrosine kinase, required for the mast cell accumulation. In our cell-based system, PMA evoked the time-dependent production of inflammatory mediators TSLP, IL-8, GM-CSF, TNF-α, and IL-6. The NTCI suppressed their production as compared to the untreated human KCs (FIG. 8D). Regarding mast cells, we also found in our most recent experimental sepsis study that the NTCI suppresses Mrgpra2a, the mouse homolog to the human gene encoding MRGPRX2, the key Mas-related G protein-coupled receptor-X2 mediating mast-cell activation in response to a broad array of stimuli. They include wasp venom and several pharmaceutical compounds associated with the IgE-independent pseudo-allergic reactions in patients.

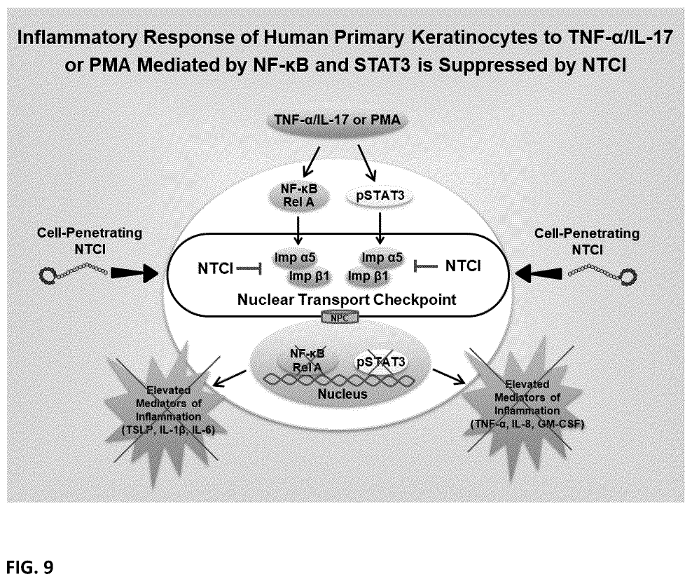

The discovery of the nuclear transport pathway mediated by NF-κB and STAT3 in human KCs responding to different inflammatory inducers (TNF-α and IL-17 or PMA), is of broader significance. It explains why the expression of at least 33 inflammatory mediators in human KCs poses a major challenge to the currently used methods of treatment that target only one or two mediators of AD and other skin diseases. We submit that inflammatory response in human KCs is chiefly mediated by the proinflammatory SRTFs (FIG. 9). These transcription factors encompass not only NF-κB and STAT3, as depicted in FIG. 9, but also other SRTFs controlled by the NTCI. They include AP1 (cFos and cJun), STAT1, STAT6, and NFATs (see FIG. 7). STAT3 and its nuclear shuttle importin α5, targeted by the NTCI as reported herein, are also involved in the mechanism of pain related to lesioned peripheral nerve. Thus, anti-inflammatory action of the NTCI extends to the cardinal signs of skin inflammation, redness, swelling, heat, and pain.

Taken together, our findings delineate the proinflammatory response of human KCs to noxious stimuli in three steps: 1. Activation of stress-responsive transcription factors, NF-κB and STAT3, among others; 2. Nuclear translocation of SRTFs to activate inflammatory regulome in the cell's nucleus; and 3. Activation of genes encoding inflammatory mediators, cytokines, chemokines, hematopoietic and vascular growth factors, intracellular signal transducers, and cell-adhesion molecules.

Importantly, the NTCI selectively inhibits these steps culminating in the nuclear translocation of larger transcription factors (MW>45 kDa), such as SRTFs and MTFs ferried by the Imp α5/Imp β1 complex. Therefore, in the presence of the NTCI, the smaller transcription factors (MW<45 kDa), which are essential to the cell's survival and maintenance, can freely translocate to the nucleus to maintain homeostasis and lifespan of cells. Moreover, the remaining importins alpha (α1, α3, α4, α6, and α7) can also ferry other nuclear proteins, including different transcription factors, during the NTCI-imposed selective nuclear blockade.

In summary, primary human skin-derived KCs are protected by the NTCI from the noxious inducers of skin inflammation through the transcriptional inhibition of the key genes involved in AD and other inflammatory skin diseases. Hence, their silencing in human KCs paves the way for the effective new treatment of AD, the most common skin disease in the world, other inflammatory skin diseases, in which injury of KCs (e.g., UV and gamma radiation) activates transcription factors NF-κB and AP1, as well as auto-inflammatory skin diseases. The NTCI can provide the protection from the exaggerated inflammatory response. Our study of human KCs and their cytoprotection, based on the selective nuclear blockade by the NTCI, adds a new perspective to the ongoing Phase 2 clinical trial of NTCI (AMTX-100 CF) for mild to moderate AD (NCT04313400).

### b) Materials and Methods

(1) The Synthesis and Purification of the Cell-Penetrating Nuclear Transport Checkpoint Inhibitor, cSN50.1 Peptide

Cell-penetrating NTCI peptide, cSN50.1 (AAVALLPAVLLALLAPCVQRKRQKLMPC, SEQ ID NO: 2) was synthesized as described elsewhere. Briefly, the peptide chain was assembled through Solid Phase Peptide Synthesis (SPPS) according to standard Fmoc chemistry protocols using an automated peptide synthesizer FOCUS XC (AAPPTec, Louisville, KY). Crude peptides were removed from the resin with a TFA cleavage cocktail and purified by dialysis against double-distilled water in 1 KDa membrane (Spectra/Por 7; Spectrum Laboratories, Rancho Dominguez, CA). The purity and structure of the final products were verified respectively by an analytical C18 RP HPLC (Beckman Coulter GOLD System, Brea, CA) and MALDI mass spectroscopy (Voyager Elite; PerSeptive Biosystems, Framingham, MA).

### (2) Cell Culture and Treatment

Human Primary Epidermal Keratinocytes (ATCC; PCS-200-011) were cultured according to the supplier's recommendations in 10 cm dishes until 80% confluent. Cells were stimulated with the mixture of TNF-α (10 ng/ml; EMD Millipore Corporation) and IL-17 (20 μg/ml; EMD Millipore Corporation) or with PMA (50 nM; Millipore-Sigma) and incubated at 37° C. in 5% CO₂. 30 minutes before challenge, cells were pretreated with cSN50.1 peptide, 10 μM (for TNF-α/IL-17) or 30 μM (for PMA). Samples of supernatant for cytokines and chemokines determination were collected at 24- and 48-hours post TNF-α/IL-17, or at 6- and 24-hours post PMA challenge.

### (3) The Nuclear Translocation of Stress-Responsive Transcription Factors (SRTFs) in Cultured Human Keratinocytes

The cells were harvested 48 hours after TNF-α/IL-17 challenge, lysed with a hypotonic buffer containing 2% NP-40, protease and phosphatase inhibitors (Roche), and washed 3 times to yield clean nuclei. Nuclear proteins were obtained by a high-salt extraction (450 mM NaCl; 4° C., 2000 rpm 30 min.). The TNF-α/IL-17-stimulated cells not treated with the NTCI (stimulated) and unstimulated cells (mock control) served as positive and negative controls, respectively. In all conditions, the cell viability was greater than 80%. The nuclear content of NF-κB RelA and phosphorylated STAT3 (pSTAT3) was determined by quantitative immunoblotting using a rabbit monoclonal anti-NF-κB p65 (RelA) antibody (Cell Signaling Technology, #8242) and rabbit polyclonal anti-pSTAT3 (Cell Signaling Technology, #9131). The rabbit monoclonal anti-Histone 3 (Cell Signaling Technology, #9715) was used to measure Histone 3 as a nuclear loading control for normalization. Li-Cor IRDye fluorescently labeled secondary antibodies were used for detection of immunoreactive bands. Immunoblots were analyzed on a Li-Cor Biosciences Odyssey Infrared Imaging System and bands were quantified by densitometry analysis using LI-COR Image Studio 3.1. Each cell-based experiment was performed in duplicate and repeated at least twice to assure experimental significance and reproducibility.

### (4) Gene Expression Assay by Real-Time Quantitative Reverse Transcription PCR

Cultured human primary keratinocytes were disrupted in the lysis buffer on ice 48 hours post TNFα/IL-17 challenge. Total RNA was isolated using NucleoSpin RNA Plus kit (Macherey-Nagel, Germany) according to the manufacturer's instructions. RNA concentration and purity were determined using a NanoDrop One spectrophotometer (Thermo Fisher Scientific). 1 μg of the obtained RNA was reverse transcribed using an iScript cDNA synthesis kit (Bio-Rad, CA). A real-time quantitative reverse transcription PCR was carried out in a 96-well plate on a QuantStudio 3 instrument using the Taqman Fast Advanced Master Mix (Thermo Fisher Scientific) according to the manufacturer's protocol. The FAM-labeled probes for analyzed genes were obtained from Thermo Fisher Scientific. The raw Ct values were converted into relative expression levels using Livak's methods (2^{ΔΔCt}) with either αTubulin gene as a reference, and the Mock Control group as a calibrator (control). Converted Ct values were used for statistical analysis.

### (5) Cytokine/Chemokine Assay

Cytokines and chemokines released by stimulated human keratinocytes were measured in a medium collected from cultured cells at indicated timepoints. Determination of TSLP level was performed by ELISA assay (Invitrogen). The levels of IL-6, TNF-α, GM-CSF (CSF2) and chemokine IL-8 (CXCL8) were determined by the cytometric bead array (CBA) assay (BD Biosciences) in the Vanderbilt University Medical Center Flow Cytometry Shared.

### (6) Statistical Analysis

The normal distribution of data sets was verified using a normal probability plot (q-q) and a Kolmogorov-Smirnov Normality Test. A statistical analysis was performed using tools built-in Prism 6 software (GraphPad). Immunoblots of SRTFs (NF-κB RelA and pSTAT3) in nuclear extracts from cultured primary human keratinocytes, and gene expression profile in human keratinocytes were analyzed by ordinary one-way ANOVA with an uncorrected Fisher's LSD test for a multiple comparison. Cytokines TSLP, IL-6, TNF-α, GM-CSF (CSF-2) and chemokine IL-8 (CXCL8) levels in the medium from cultured human keratinocytes were evaluated by the two-way ANOVA using an uncorrected Fisher's LSD test for multiple comparison. The data is presented as a mean±SEM. p values of <0.05 were considered significant.

### D. References

Aksentijevich, I. et al. An autoinflammatory disease with deficiency of the interleukin-1-receptor antagonist. N. Engl. J. Med. 360 (23), 2426-2437 (2009).
Bashir M M, Sharma M R, Werth V P. TNF-alpha production in the skin. Arch Dermatol Res 2009; 301 (1): 87-91.
Beck, L. A. et al. Dupilumab treatment in adults with moderate-to-severe atopic dermatitis. N. Engl. J. Med. 371 (2), 130-139 (2014).
Ben-Yaakov, K. et al. Axonal transcription factors signal retrogradely in lesioned peripheral nerve. EMBO J. 31 (6), 1350-1363 (2012).
Briot, A. et al. Kallikrein 5 induces atopic dermatitis-like lesions through PAR2-mediated thymic stromal lymphopoietin expression in Netherton syndrome. J. Exp. Med. 206 (5), 1135-1147 (2009).
Bromley, S. K., Larson, R. P., Ziegler, S. F. & Luster, A. D. IL-23 induces atopic dermatitis-like inflammation instead of psoriasis-like inflammation in CCR2-deficient mice. PLoS ONE 8 (3), e58196 (2013).
Brunner, P. M., Guttman-Yassky, E. & Leung, D. Y. The immunology of atopic dermatitis and its reversibility with broad-spectrum and targeted therapies. J. Allergy Clin. Immunol. 139 (4S), S65-S76 (2017).
DiGiandomenico, A. et al. The “genomic storm” induced by bacterial endotoxin is calmed by a nuclear transport modifier that attenuates localized and systemic inflammation. PLoS ONE 9 (10), e110183 (2014).
Dougan M, Dranoff G, Dougan S K. GM-CSF, IL-3, and IL-5 Family of Cytokines: Regulators of Inflammation. Immunity 2019; 50 (4): 796-811.
Duvigneau J C, Luis A, Gorman A M, Samali A, Kaltenecker D, Moriggl R, et al. Crosstalk between inflammatory mediators and endoplasmic reticulum stress in liver diseases. Cytokine 2019; 124:154577.
Ebner, S. et al. Thymic stromal lymphopoietin converts human epidermal Langerhans cells into antigen-presenting cells that induce proallergic T cells. J. Allergy Clin. Immunol. 119 (4), 982-990 (2007).
Fousek K, Horn L A, Palena C. Interleukin-8: A chemokine at the intersection of cancer plasticity, angiogenesis, and immune suppression. Pharmacol Ther 2021; 219:107692.
Furue, M. Regulation of skin barrier function via competition between AHR Axis versus IL-13/IL-4JAKSTAT6/STAT3 Axis: pathogenic and therapeutic implications in atopic dermatitis. J. Clin. Med. 9 (11), 3741 (2020).
Gabay, C. & Kushner, I. Acute-phase proteins and other systemic responses to inflammation. N. Engl. J. Med. 340 (6), 448-454 (1999).
Gilhar, A. et al. Mouse models of atopic dermatitis: A critical reappraisal. Exp. Dermatol. 30 (3), 319-336 (2021).
Gottlieb, A. B. Therapeutic options in the treatment of psoriasis and atopic dermatitis. J. Am. Acad. Dermatol. 53 (1 Suppl 1), S3-16 (2005).
Grundy, S. M. et al. Definition of metabolic syndrome: Report of the National Heart, Lung, and Blood Institute/American Heart Association conference on scientific issues related to definition. Arterioscler. Thromb. Vasc. Biol. 24 (2), e13-18 (2004).
Hawiger, J. & Zienkiewicz, J. Decoding inflammation, its causes, genomic responses, and emerging countermeasures. Scand. J. Immunol. 90 (6), e12812 (2019).
Hawiger, J. & Zienkiewicz, J. Endothelial cytoprotection in metabolic and microbial inflammation underlying cardiovascular diseases. Cardiovasc. Res. (2022)—under review
Jiang Y, Tsoi L C, Billi A C, Ward N L, Harms P W, Zeng C, et al. Cytokinocytes: the diverse contribution of keratinocytes to immune responses in skin. JCI Insight 2020; 5 (20).
Kaufman, B. P., Guttman-Yassky, E. & Alexis, A. F. Atopic dermatitis in diverse racial and ethnic groups-Variations in epidemiology, genetics, clinical presentation and treatment. Exp. Dermatol. 27 (4), 340-357 (2018).
Kim, D., Kobayashi, T. & Nagao, K. Research techniques made simple: Mouse models of atopic dermatitis. J. Invest. Dermatol. 139 (5), 984-990.el (2019).
Kubo, A., Nagao, K. & Amagai, M. Epidermal barrier dysfunction and cutaneous sensitization in atopic diseases. J. Clin. Invest. 122 (2), 440-447 (2012).
Lehmann, H. S., Heaton, T., Mallon, D. & Holt, P. G. Staphylococcal enterotoxin-B-mediated stimulation of interleukin-13 production as a potential aetiologic factor in eczema in infants. Int. Arch. Allergy Immunol. 135 (4), 306-312 (2004).
Li, M. et al. Induction of thymic stromal lymphopoietin expression in keratinocytes is necessary for generating an atopic dermatitis upon application of the active vitamin D3 analogue MC903 on mouse skin. J. Invest. Dermatol. 129 (2), 498-502 (2009).
Li, M. et al. Topical vitamin D3 and low-calcemic analogs induce thymic stromal lymphopoietin in mouse keratinocytes and trigger an atopic dermatitis. Proc. Natl. Acad. Sci. USA. 103 (31), 11736-11741 (2006).
Liu Y, Zienkiewicz J, Qiao H, Gibson-Corley K N, Boyd K L, Veach R A, et al. Genomic control of inflammation in experimental atopic dermatitis. Scientific reports 2022; 12 (1): 18891.
Liu, D. et al. Suppression of staphylococcal enterotoxin B-induced toxicity by a nuclear import inhibitor. J. Biol. Chem. 279 (18), 19239-19246 (2004).
Liu, D., Zienkiewicz, J., DiGiandomenico, A. & Hawiger, J. Suppression of acute lung inflammation by intracellular peptide delivery of a nuclear import inhibitor. Mol. Ther. 17 (5), 796-802 (2009).
Liu, Y. et al. Hyperlipidemic hypersensitivity to lethal microbial inflammation and its reversal by selective targeting of nuclear transport shuttles. Sci. Rep. 11 (1), 11907 (2021).
Liu, Y. et al. Nuclear transport modulation reduces hypercholesterolemia, atherosclerosis, and fatty liver. J. Am. Heart Assoc. 2 (2), e000093 (2013).
Marković I, Savvides S N. Modulation of Signaling Mediated by TSLP and IL-7 in Inflammation, Autoimmune Diseases, and Cancer. Frontiers in immunology 2020; 11:1557.
Marschall, P. et al. Dual function of Langerhans cells in skin TSLP-promoted T_{FH} differentiation in mouse atopic dermatitis. J. Allergy Clin. Immunol. 147 (5), 1778-1794 (2021).
McNeil B D, Pundir P, Meeker S, Han L, Undem B J, Kulka M, et al. Identification of a mast-cell-specific receptor crucial for pseudo-allergic drug reactions. Nature 2015; 519 (7542): 237-41.
Nutten, S. Atopic dermatitis: Global epidemiology and risk factors. Ann. Nutr. Metab. 66 (Suppl 1), 8-16 (2015).
Ong, P. Y. et al. Endogenous antimicrobial peptides and skin infections in atopic dermatitis. N. Engl. J. Med. 347 (15), 1151-1160 (2002).
Poli, V. The role of C/EBP isoforms in the control of inflammatory and native immunity functions. J. Biol. Chem. 273 (45), 29279-29282 (1998).
Qiao H, Zienkiewicz J, Liu Y, Hawiger J. Activation of Thousands of Genes in the Lungs and Kidneys by Sepsis is Countered by the Selective Nuclear Blockade. Front Immunol 2023; 14:1221102
Rijken F, Bruijnzeel P L, van Weelden H, Kiekens R C. Responses of black and white skin to solar-simulating radiation: differences in DNA photodamage, infiltrating neutrophils, proteolytic enzymes induced, keratinocyte activation, and IL-10 expression. J Invest Dermatol 2004; 122 (6): 1448-55.
Roy S, Chompunud Na Ayudhya C, Thapaliya M, Deepak V, Ali H. Multifaceted MRGPRX2: New insight into the role of mast cells in health and disease. J Allergy Clin Immunol 2021; 148 (2): 293-308.
Schoepe, S., Schacke, H., May, E. & Asadullah, K. Glucocorticoid therapy-induced skin atrophy. Exp. Dermatol. 15 (6), 406-420 (2006).
Schwartz, C. et al. Spontaneous atopic dermatitis in mice with a defective skin barrier is independent of ILC2 and mediated by IL-1β. Allergy 74 (10), 1920-1933 (2019).
Siiskonen, H. & Harvima, I. Mast cells and sensory nerves contribute to neurogenic inflammation and pruritus in chronic skin inflammation. Front Cell Neurosci. 13, 422 (2019).
Silverberg, J. I. & Paller, A. S. Association between eczema and stature in 9 US population-based studies. JAMA Dermatol. 151 (4), 401-409 (2015).
Steinhoff, M., Schmelz, M., Szabo, I. L. & Oaklander, A. L. Clinical presentation, management, and pathophysiology of neuropathic itch. Lancet Neurol. 17 (8), 709-720 (2018).
Theoharides, T. C. et al. IL-33 augments substance P-induced VEGF secretion from human mast cells and is increased in psoriatic skin. Proc. Natl. Acad. Sci. USA. 107 (9), 4448-4453 (2010).
Torgerson, T. R., Colosia, A. D., Donahue, J. P., Lin, Y. Z. & Hawiger, J. Regulation of NF-kappa B, AP-1, NFAT, and STAT1 nuclear import in T lymphocytes by noninvasive delivery of peptide carrying the nuclear localization sequence of NF-kappa B p50. J. Immunol. 161 (11), 6084-6092 (1998).
Waskow, C., Bartels, S., Schlenner, S. M., Costa, C. & Rodewald, H. R. Kit is essential for PMA-inflammation-induced mast-cell accumulation in the skin. Blood 109 (12), 5363-5370 (2007).
Weidinger, S., Beck, L. A., Bieber, T., Kabashima, K. & Irvine, A. D. Atopic dermatitis. Nat. Rev. Dis. Primers. 4 (1), 1 (2018).
Wilson, S. R. et al. The epithelial cell-derived atopic dermatitis cytokine TSLP activates neurons to induce itch. Cell 155 (2), 285-295 (2013).
Zienkiewicz, J., Armitage, A. & Hawiger, J. Targeting nuclear import shuttles, importins/karyopherins alpha by a peptide mimicking the NFkappaB1/p50 nuclear localization sequence. J. Am. Heart Assoc. 2 (5), e000386 (2013).

What is claimed is:

1. A method of treating/inhibiting/reducing an inflammatory skin disease or inflammatory skin disorder caused by microbial, allergic, autoimmune, constitutive, metabolic, neoplastic and/or physical insults in a subject comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates a skin disease or inflammatory skin disorder; and c) administering to the subject with elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111, a therapeutically effective amount of a composition comprising a Nuclear Transport Checkpoint Inhibitor (NTCI).

2. The method of claim 1, wherein the NTCI comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

3. The method of claim 1, wherein the inflammatory skin disease or inflammatory skin disorder is caused by microbial agents, autoimmune disorder, alloimmune disorder, allergic process, autoinflammatory disorder, metabolic process, neoplastic/oncogenic disease, or physical insults that are mediated by inflammation.

4. The method of claim 3, wherein the inflammatory skin disease is caused by a microbial infection selected from the group consisting of a viral infection, a bacterial infection, a fungal infection, or a parasitic infection; wherein when the microbial infection is a bacterial infection, the bacteria causing the bacterial infection is not Bacillus anthracis.

5. The method of claim 4, further comprising administering to the subject an antimicrobial agent.

6. The method of claim 4, wherein the NTCI composition comprises the anti-microbial agent.

7. The method of claim 3, wherein the inflammatory skin disease is caused by a metabolic process/insult.

8. The method of claim 7, wherein the metabolic process is selected from the group consisting of Gout, Skin Atrophy, Skin Aging, Xanthelasma, metabolic syndrome, diabetes mellitus, obesity, Gaucher's disease, Phenylketonuria (PKU), Maple syrup urine disease (MSUD), fatty liver, hypercholesterolemia, hypertriglyceridemia, hyperthyroidism, hypothyroidism, dyslipidemia, hypolipidemia, seborrheic dermatitis, and galactosemia.

9. The method of claim 3, wherein the inflammatory skin disorder is caused by physical injury selected from the group consisting of abrasion, puncture, laceration, contusion, blunt force trauma, ischemia, surgery, bedsores, electric burn, sunburn, chemical burn, high temperature burn, low temperature burn, radiation injury, and skin aging.

10. A method of assessing a treatment regimen or a subjects' responsiveness to a treatment regimen for an inflammatory skin disease or inflammatory skin disorder caused by a microbial, allergic, autoimmune, constitutive (auto-inflammatory), metabolic, neoplastic and/or physical insults in a subject comprising a) obtaining a tissue sample from the subject; b) measuring the tissue sample for elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111; wherein elevated expression or activity of one or more of TSLP, IL-1β, IL-4, IL-4Rα, IL-6, IL-8, IL-10, IL-13, IL-17, IL-18, IL-23α, IL-33, IFN-γ, TNF-α, MCP-1, GM-CSF, RANTES, VEGF A, VCAM-1, COX-2, iNOS, MRGPRX2, SREBf1, Srebf2, Hmgcr, Fasn, Acly, Pcsk9, and/or Npac111 indicates that the treatment regimen is not effective against the inflammatory skin disease or inflammatory skin disorder caused by a microbial, allergic, autoimmune, constitutive, metabolic, neoplastic and/or physical insults or the subject is not responsive to the treatment regimen.

11. The method of claim 10, wherein the treatment regimen comprises a composition comprising a nuclear transport checkpoint inhibitor (NTCI).

12. The method of claim 11, wherein the NTCI comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

13. The method of claim 10, wherein the inflammatory skin disease or inflammatory skin disorder is caused by microbial agents, autoimmune disorder, alloimmune disorder, allergic process, autoinflammatory disorder, metabolic process, neoplastic/oncogenic disease, or physical insults that are mediated by inflammation.

14. The method of claim 13, wherein the inflammatory skin disease is caused by a microbial infection selected from the group consisting of a viral infection, a bacterial infection, a fungal infection, or a parasitic infection; wherein when the microbial infection is a bacterial infection, the bacteria causing the bacterial infection is not Bacillus anthracis.

15. The method of claim 14, further comprising administering to the subject an antimicrobial agent.

16. The method of claim 15, wherein the NTCI composition comprises the anti-microbial agent.

17. The method of claim 13, wherein the inflammatory skin disease is caused by a metabolic process/insult selected from the group consisting of Gout, Skin Aging, Xanthelasma, metabolic syndrome, diabetes mellitus, obesity, Gaucher's disease, Phenylketonuria (PKU), Maple syrup urine disease (MSUD), fatty liver, hypercholesterolemia, hypertriglyceridemia, hyperthyroidism, hypothyroidism, dyslipidemia, hypolipidemia, seborrheic dermatitis, and galactosemia.

18. The method of claim 13, wherein the inflammatory skin disorder is caused by physical injury selected from the group consisting of, abrasion, puncture, laceration, contusion, blunt force trauma, ischemia, surgery, graft versus host disease after transplantation, bedsores, electric burn, sunburn, chemical burn, high temperature burn, low temperature burn, radiation injury, and skin aging.


SEQUENCE LISTING (41 sequences)

SEQ ID NO: 1 - protein, 29 aa, synthetic construct
  REGION 1..29: note = Description of sequence: Peptide, name of peptide: cSN50
AAVALLPAVLLALLAPCYVQRKRQKLMPC

SEQ ID NO: 2 - protein, 28 aa, synthetic construct
  REGION 1..28: note = Description of sequence: Peptide, name of peptide: cSN50.1
AAVALLPAVLLALLAPCVQRKRQKLMPC

SEQ ID NO: 3 - protein, 29 aa, synthetic construct
  REGION 1..29: note = Description of sequence: Peptide, name of peptide: Nuclear Transport Modifier (NTM)
  VARIANT 1..4: note = X is any known amino acid
  VARIANT 8..10: note = X is any known amino acid
  VARIANT 17..20: note = X is any known amino acid
  VARIANT 26..29: note = X is any known amino acid
  VARIANT 1..4: note = Position may be absent
  VARIANT 8..10: note = Position may be absent
  VARIANT 17..20: note = Position may be absent
  VARIANT 26..29: note = Position may be absent
XXXXLLPXXLLALLAPXXXQRKRQKXXXX

SEQ ID NO: 4 - protein, 29 aa, synthetic construct
  REGION 1..29: note = Description of sequence: Peptide, name of peptide: Nuclear Transport Modifier (NTM)
  VARIANT 1..4: note = X is any known amino acid
  VARIANT 8..10: note = X is any known amino acid
  VARIANT 18..20: note = X is any known amino acid
  VARIANT 26..28: note = X is any known amino acid
  VARIANT 1..4: note = Position may be absent
  VARIANT 8..10: note = Position may be absent
  VARIANT 18..20: note = Position may be absent
  VARIANT 26..28: note = Position may be absent
XXXXLLPXXLLALLAPCXXQRKRQKXXXC

SEQ ID NO: 5 - protein, 28 aa, synthetic construct
  REGION 1..28: note = Description of sequence: Peptide, name of peptide: Nuclear Transport Modifier (NTM)
  VARIANT 1..4: note = X is any known amino acid
  VARIANT 8..10: note = X is any known amino acid
  VARIANT 18: note = X is any known amino acid
  VARIANT 25..27: note = X is any known amino acid
  VARIANT 1..4: note = Position may be absent
  VARIANT 8..10: note = Position may be absent
  VARIANT 18: note = Position may be absent
  VARIANT 25..27: note = Position may be absent
XXXXLLPXXLLALLAPCXQRKRQKXXXC

SEQ ID NO: 6 - protein, 29 aa, synthetic construct
  REGION 1..29: note = Description of sequence: Peptide, name of peptide: Nuclear Transport Modifier (NTM)
  VARIANT 1..4: note = X is any known amino acid
  VARIANT 8..10: note = X is any known amino acid
  VARIANT 17..20: note = X is any known amino acid
  VARIANT 26..29: note = X is any known amino acid
  VARIANT 1..4: note = Position may be absent
  VARIANT 8..10: note = Position may be absent
  VARIANT 17..20: note = Position may be absent
  VARIANT 26..29: note = Position may be absent
XXXXLLPXXLLAVLAPXXXQRKRQKXXXX

SEQ ID NO: 7 - protein, 28 aa, synthetic construct
  REGION 1..28: note = Description of sequence: Peptide, name of peptide: Nuclear Transport Modifier (NTM)
AAVALLPAVLLAVLAPCVQRKRQKLMPC

SEQ ID NO: 8 - protein, 27 aa, synthetic construct
  REGION 1..27: note = Description of sequence: Peptide, name of peptide: Nuclear Transport Modifier (NTM)
  VARIANT 1..4: note = X is any known amino acid
  VARIANT 8..10: note = X is any known amino acid
  VARIANT 17..20: note = X is any known amino acid
  VARIANT 26..27: note = X is any known amino acid
  VARIANT 1..4: note = Position may be absent
  VARIANT 8..10: note = Position may be absent
  VARIANT 17..20: note = Position may be absent
  VARIANT 26..27: note = Position may be absent
XXXXLLPXXLLAVLAPXXXQRDEQKXX

SEQ ID NO: 9 - protein, 28 aa, synthetic construct
  REGION 1..28: note = Description of sequence: Peptide, name of peptide: Nuclear Transport Modifier (NTM)
AAVALLPAVLLAVLAPCVQRDEQKLMPC

SEQ ID NO: 10 - protein, 10 aa, synthetic construct
  REGION 1..10: note = Description of sequence: Peptide, name of peptide: N50
VQRKRQKLMP

SEQ ID NO: 11 - protein, 10 aa, synthetic construct
  REGION 1..10: note = Description of sequence: Peptide, name of peptide: N50M
VQRDEQKLMP

SEQ ID NO: 12 - protein, 12 aa, synthetic construct
  REGION 1..12: note = Description of sequence: Peptide, name of peptide: cN50.1
CVQRKRQKLMPC

SEQ ID NO: 13 - protein, 26 aa, synthetic construct
  REGION 1..26: note = Description of sequence: Peptide, name of peptide: SN50
AAVALLPAVLLALLAPVQRKRQKLMP

SEQ ID NO: 14 - protein, 7 aa, synthetic construct
  REGION 1..7: note = Description of sequence: Peptide, name of peptide: SSHR-1
AAVALLP

SEQ ID NO: 15 - protein, 9 aa, synthetic construct
  REGION 1..9: note = Description of sequence: Peptide, name of peptide: SSHR-2
AVLLALLAP

SEQ ID NO: 16 - protein, 27 aa, synthetic construct
  REGION 1..27: note = Description of sequence: Peptide, name of peptide: cSN50.1 beta
AVALLPAVLLALLAPCVQRDEQKLMPC

SEQ ID NO: 17 - protein, 16 aa, synthetic construct
  REGION 1..16: note = Description of sequence: Peptide, name of peptide: SSHR
AAVALLPAVLLALLAP

SEQ ID NO: 18 - protein, 32 aa, synthetic construct
  REGION 1..32: note = Description of sequence: Peptide, name of peptide: SSIB
AAVALLPAVLLALLAPRRRRIEVNVELRKAKK

SEQ ID NO: 19 - protein, 34 aa, synthetic construct
  REGION 1..34: note = Description of sequence: Peptide, name of peptide: SI-1
AAVALLPAVLLALLAPRRRRIEVNVELRKAKKDD

SEQ ID NO: 20 - protein, 34 aa, synthetic construct
  REGION 1..34: note = Description of sequence: Peptide, name of peptide: SI-3
AAVALLPAVLLALLAPRRQRNEVVVELRKNKRDE

SEQ ID NO: 21 - protein, 34 aa, synthetic construct
  REGION 1..34: note = Description of sequence: Peptide, name of peptide: SI-4
AAVALLPAVLLALLAPRRHRNEVTVELRKNKRDE

SEQ ID NO: 22 - protein, 34 aa, synthetic construct
  REGION 1..34: note = Description of sequence: Peptide, name of peptide: SI-5
AAVALLPAVLLALLAPRRRREEEGLQLRKQKREE

SEQ ID NO: 23 - protein, 34 aa, synthetic construct
  REGION 1..34: note = Description of sequence: Peptide, name of peptide: SI-7
AAVALLPAVLLALLAPRRRREEEGIQLRKQKREQ

SEQ ID NO: 24 - protein, 28 aa, synthetic construct
  REGION 1..28: note = Description of sequence: Peptide, name of peptide: cSIB
AAVALLPAVLLALLAPCTEMRRRRIEVC

SEQ ID NO: 25 - protein, 37 aa, synthetic construct
  REGION 1..37: note = Description of sequence: Peptide, name of peptide: SAR1
AAVALLPAVLLALLAPVELRKAKKDDQMLKRRNVSSF

SEQ ID NO: 26 - protein, 37 aa, synthetic construct
  REGION 1..37: note = Description of sequence: Peptide, name of peptide: SAR3
AAVALLPAVLLALLAPVELRKNKRDEHLLKRRNVPHE

SEQ ID NO: 27 - protein, 37 aa, synthetic construct
  REGION 1..37: note = Description of sequence: Peptide, name of peptide: SAR4
AAVALLPAVLLALLAPVELRKNKRDEHLLKKRNVPQE

SEQ ID NO: 28 - protein, 37 aa, synthetic construct
  REGION 1..37: note = Description of sequence: Peptide, name of peptide: SAR5
AAVALLPAVLLALLAPLQLRKQKREEQLFKRRNVATA

SEQ ID NO: 29 - protein, 37 aa, synthetic construct
  REGION 1..37: note = Description of sequence: Peptide, name of peptide: SAR7
AAVALLPAVLLALLAPIQLRKQKREQQLFKRRNVELI

SEQ ID NO: 30 - protein, 29 aa, synthetic construct
  REGION 1..29: note = Description of sequence: Peptide, name of peptide: cSAR1-C
AAVALLPAVLLALLAPCVELRKAKKDDQC

SEQ ID NO: 31 - protein, 29 aa, synthetic construct
  REGION 1..29: note = Description of sequence: Peptide, name of peptide: cSAR3-C
AAVALLPAVLLALLAPCVELRKNKRDEHC

SEQ ID NO: 32 - protein, 29 aa, synthetic construct
  REGION 1..29: note = Description of sequence: Peptide, name of peptide: cSAR5-C
AAVALLPAVLLALLAPCLQLRKQKREEQC

SEQ ID NO: 33 - protein, 29 aa, synthetic construct
  REGION 1..29: note = Description of sequence: Peptide, name of peptide: cSAR7-C
AAVALLPAVLLALLAPCIQLRKQKREQQC

SEQ ID NO: 34 - protein, 29 aa, synthetic construct
  REGION 1..29: note = Description of sequence: Peptide, name of peptide: cSAR1-N
AAVALLPAVLLALLAPCQMLKRRNVSSFC

SEQ ID NO: 35 - protein, 29 aa, synthetic construct
  REGION 1..29: note = Description of sequence: Peptide, name of peptide: cSAR3-N
AAVALLPAVLLALLAPCHLLKRRNVPHEC

SEQ ID NO: 36 - protein, 29 aa, synthetic construct
  REGION 1..29: note = Description of sequence: Peptide, name of peptide: cSAR4-N
AAVALLPAVLLALLAPCHLLKKRNVPQEC

SEQ ID NO: 37 - protein, 29 aa, synthetic construct
  REGION 1..29: note = Description of sequence: Peptide, name of peptide: cSAR5-N
AAVALLPAVLLALLAPCQLFKRRNVATAC

SEQ ID NO: 38 - protein, 29 aa, synthetic construct
  REGION 1..29: note = Description of sequence: Peptide, name of peptide: cSAR7-N
AAVALLPAVLLALLAPCQLFKRRNVELIC

SEQ ID NO: 39 - protein, 37 aa, synthetic construct
  REGION 1..37: note = Description of sequence: Peptide, name of peptide: Nuclear Transport Modifier (NTM)
AAVALLPAVXLAXXAPVELRKNKRDEHLLKRRNVPHE

SEQ ID NO: 40 - protein, 26 aa, synthetic construct
  REGION 1..26: note = Description of sequence: Peptide, name of peptide: SM12
AAVALLPAVLLALLAPVQRDEQKLMP

SEQ ID NO: 41 - protein, 28 aa, synthetic construct
  REGION 1..28: note = Description of sequence: Peptide, name of peptide: importin alpha-selective NTM
  VARIANT 10: note = X is any known amino acid
  VARIANT 13..14: note = X is any known amino acid
AAVALLPAVXLAXXAPCVQRKRQKLMPC